(19) United States
(12) Patent Application Publication
(10) Pub. No.: US 2025/0081946 A1
(43) Pub. Date: Mar. 13, 2025

(54) INSECT-LARVAE BREEDING APPARATUS HAVING AN ACTIVITY-SENSOR DEVICE

(71) Applicant: FARMINSECT GMBH, Bergkirchen (DE)

(72) Inventors: Wolfgang WESTERMEIER, Bergkirchen (DE); Thomas KÜHN, Bergkirchen (DE)

(21) Appl. No.: 18/723,827

(22) PCT Filed: Dec. 21, 2022
(86) PCT No.: PCT/EP2022/087375

§ 371 Date: Jun. 24, 2024

(30) Foreign Application Priority Data
Dec. 22, 2021 (DE) 10 2021 134 239.2

(51) Int. Cl. A01K 67/033; A01K 29/00

(52) U.S. Cl. CPC A01K 67/30; A01K 29/00

(57) ABSTRACT

A rearing device for black soldier fly larvae includes a first insect fattening container that accommodates a first insect larvae cohort for fattening, an activity sensor device, and a processing unit. The activity sensor device detects a humidity measurement value at an insect fattening container humidity measurement point and provides the value to the processing unit. The insect fattening container humidity measurement point is in a central section of the insect fattening container. The activity sensor device also detects a temperature measurement value at an insect fattening container temperature measurement point and provides the value to the processing unit. The insect fattening container temperature measurement point is in the central portion of the first insect fattening container. The processing unit is designed to process the measured humidity value and the measured temperature value and to determine the activity of the black soldier fly larvae based on the processing.

The invention relates to an insect larvae rearing device, in particular a rearing device for larvae of the black soldier fly.

The increasing demand for protein for livestock farming and for a growing world population requires alternative protein sources. Insect larvae have a high protein content, can be fed with organic waste, and are significantly more climate-friendly to rear than conventional protein sources. Insect larvae are therefore suitable for supplementing or completely replacing protein-rich feed for livestock farming, such as fishmeal.

In addition to providing an alternative source of protein for their livestock, farmers with livestock farms also have the opportunity to utilise their own organic waste by using insect larvae. It therefore makes sense to carry out a large part of the rearing directly on the farmer's premises, so that the farmer benefits from the insect larvae not only in the form of protein-rich feed, but also in the form of natural waste processors.

Until now, the rearing of insect larvae has largely been carried out manually. Rearing is understood here to mean in particular the extraction of larvae from eggs. A high hatching rate and a large number of healthy larvae, which can then be processed into animal feed, is always desirable. In some cases, larvae monitoring is installed to assess the condition of the larvae. From the evaluation of the larval condition, conclusions can be drawn about an optimal harvest time, and recommendations for action can be derived, such as adjustments to feeding or the climate in the insect larvae rearing facility.

With increasing decentralisation, it is desirable to support the evaluation of larval condition by systems in order to optimise harvest yield and achieve improved feed quality of the animal feed obtained from the insect larvae.

For example, the modular system of document WO 2019/053439 A2 comprises sensors and cameras that enable remote monitoring of the larvae. A camera and a Bluetooth-enabled thermocouple are each disposed in a rearing tray, whereby the camera is configured to visually assess the condition of the larvae and the thermocouple is configured to measure the temperature of the insect larvae. The main disadvantages of this solution are the complex set-up of the equipment and the fact that the condition of the larvae is still assessed visually.

The object of the invention is to provide a device which is improved with respect to an assessment of the activity of the insect larvae for assessing a larval condition.

The object is achieved in an insect larvae rearing device of the type mentioned above by a first insect fattening container configured to accommodate a first insect larvae cohort for fattening, an activity sensor device, and a processing unit. The activity sensor device is configured to detect at least one first humidity measurement value at at least one first humidity measuring point and to provide it to the processing unit. The first humidity measuring point is preferably disposed in a central portion of the insect fattening container.

In addition, the activity sensor device is configured to detect at least one first temperature measurement value at at least one first temperature measuring point and to provide it to the processing unit. The first temperature measuring point is preferably disposed in the central portion of the insect fattening container.

The processing unit is designed to process the detected first humidity measurement value and the detected first temperature measurement value and to determine the activity of the insect larvae based on the processing.

The activity of the insect larvae cohort within the first insect fattening container increases as the stage of development progresses. The distribution of the first insect larvae cohort within the first insect fattening container depends on the activity and consequently the developmental state of the insect larvae cohort. Repeated observation has shown that the larvae of the black soldier fly are essentially evenly distributed within the insect fattening container at the beginning of a fattening process, grouping together in a central portion of the insect fattening container as activity increases and subsequently also grouping together at the edges of the insect fattening container. In the case of a cuboid insect fattening container with a rectangular base, the insect larvae prefer to group together in the corners of the insect fattening container. As a result, particular sections of the insect fattening container are occupied or unoccupied by insect larvae at different times during the development of the insect larvae. The activity sensor device determines the activity of the insect larvae cohort, preferably without optical detection. This means that the activity sensor device preferably has no optical sensor, camera or similar devices for optical detection.

By means of the activity sensor device according to the invention and the associated arrangement of the measuring point or measuring points, a relevant section of the insect fattening container can be measured for purposes of activity determination and, as a result, it is possible to determine the activity of the insect larvae cohort. The measurement values are preferably used to locate the insect larvae inside the insect fattening container. It is preferably also possible to detect further temperature and/or humidity measuring points by means of the activity sensor device. Based on the measurement values, the position of the insect larvae within the insect fattening container is determined.

In a particularly preferred refinement, the activity sensor device is configured to detect at least one second humidity measurement value at a second humidity measuring point and/or at least one third humidity measurement value at a third humidity measuring point and to provide these to the processing unit. The second humidity measuring point is preferably laterally spaced from the first humidity measuring point and the third humidity measuring point is preferably laterally spaced from both the first humidity measuring point and the second humidity measuring point.

The activity sensor device is also preferably configured to detect at least one second temperature measurement value at a second temperature measuring point and/or at least one third temperature measurement value at a third temperature measuring point and to provide it to the processing unit. The second temperature measuring point is preferably laterally spaced from the first temperature measuring point and the third temperature measuring point is preferably laterally spaced from both the first temperature measuring point and the second temperature measuring point.

By means of this preferred arrangement of the activity sensor device, it is possible to better measure relevant sections of the insect fattening container for purposes of determining the activity of the larvae. The laterally spaced positioning of the measuring points means that the insect larvae can be reliably located within the insect fattening container.

In a preferred refinement, the second humidity measuring point is disposed on or adjacent to a side wall of the first insect fattening container. The second temperature measuring point is preferably disposed on or adjacent to a side wall of the first insect fattening container. It is preferable that the third humidity measuring point is disposed at an edge, particularly preferably at a corner, of the first insect fattening container. The third temperature measuring point is also preferably disposed at an edge, particularly preferably at a corner of the first insect fattening container. By positioning the measuring points in a central portion, on and/or adjacent to a side wall and a corner of the first insect fattening container, the insect larvae can be located particularly reliably in these sections of the first insect fattening container. These are also the sections of the first insect fattening container where the insect larvae group together during the fattening process, as has been shown by repeated observation. It is preferable that further humidity measuring points and/or temperature measuring points are disposed in the first insect fattening container. The further humidity measuring points and/or temperature measuring points are preferably disposed on or adjacent to side walls and/or corners of the insect fattening container. In a preferred refinement, humidity measuring points and/or temperature measuring points are disposed on all side walls of the insect fattening container. In a preferred refinement, humidity measuring points and/or temperature measuring points are disposed at all corners of the insect fattening container.

It is preferred that the first humidity measuring point is disposed adjacent to the first temperature measuring point, the second humidity measuring point is disposed adjacent to the second temperature measuring point, and/or the third humidity measuring point is disposed adjacent to the third temperature measuring point. In this embodiment, both a temperature measurement value and a humidity measurement value are detected at the sections relevant for determining the activity.

Preferably, a first humidity sensor is disposed at the first humidity measuring point, a second humidity sensor at the second humidity measuring point, and/or a third humidity sensor at the third humidity measuring point. It is particularly preferable that the first, second, and/or third humidity sensors are capacitive humidity sensors.

In a preferred refinement, a first temperature sensor is disposed at the first temperature measuring point, a second temperature sensor at the second temperature measuring point and/or a third temperature sensor at the third temperature measuring point.

It is further preferred that the insect larvae rearing device comprises a second insect fattening container configured to accommodate a second insect larvae cohort for fattening. In this embodiment, it is preferable for the activity sensor device to detect measurement values in an analogous manner to the detection of measurement values within the first insect fattening container. In addition, further insect fattening containers, which are configured to accommodate further insect larvae cohorts for fattening, may be provided with an analogous measuring device. For the positioning of the measurement values within the first, the second, and the other insect fattening containers, please refer to the above description regarding the first insect fattening container.

The first, the second, and the optional further insect fattening containers are disposed vertically on top of each other, for example, but also horizontally next to each other or both vertically and horizontally next to each other in the insect larvae rearing device. It is advantageous if the insect larvae rearing device has a rearing area closed off from an environment, wherein the first, the second, and the other insect fattening containers are disposed in the area, and wherein the air condition within the rearing area is known, for example is determined by sensors.

The sensor device can also be installed in a rearing device for insect larvae of a species other than the black soldier fly. To this end, it can be observed how the corresponding insect larvae genus is distributed and grouped within an insect fattening container with increasing activity. The arrangement of the measuring points should be such that suitable sections are measured to determine the activity of the corresponding insect larvae species.

The second invention further comprises a method for determining an activity of insect larvae by means of an insect larvae rearing device, comprising the steps of: Filling an insect fattening container with insect larvae with the addition of fattening substrate at the beginning of a fattening phase and processing detected first, second, and third humidity measurement values and detected first, second, and third temperature measurement values by means of a processing unit at a first point in time t1. It is particularly preferable that the fattening substrate to be added comprises a proportion of water. The fattening substrate to be added preferably comprises a proportion of water-binding substances. The fattening substrate to be added preferably includes a proportion of nutrients. The proportion of water in the fattening substrate to be added is preferably in the range of 0% to 90%. A proportion of water-binding substances in the fattening substrate to be added is preferably in the range of 10% to 100%. The proportion of nutrients in the fattening substrate to be added is preferably in the range of 0% to 100%. Water-binding substances are, for example, wheat bran, water-binding gels, or other water-binding elements. The consistency of the fattening substrate is preferably porridge-like when added. During the fattening process, the proportion of water in the fattening substrate decreases. As a result, the consistency of the fattening substrate changes and it becomes free-flowing and portionable.

Processing the detected measurement values comprises several sub-steps. In a first sub-step, the detected first humidity measurement value at time t1 is compared with a first humidity reference value at time t1, the detected second humidity measurement value at time t1 is compared with a second humidity reference value at time t1, the detected third humidity measurement value at time t1 is compared with a third humidity reference value at time t1, the detected first temperature measurement value at time t1 is compared with a first temperature reference value at time t1, the detected second temperature measurement value at time t1 is compared with a second temperature reference value at time t1, and/or the detected third temperature measurement value at time t1 is compared with a third temperature reference value at time t1.

The first, second, and/or third humidity reference values at time t1 may be different reference values or an identical reference value. The respective humidity reference values at time t1 are preferably obtained from repeated observations of humidity measurement values of the fattening substrate at time t1 and are stored in the processing unit or can be retrieved therefrom. The consistency of the fattening substrate is preferably porridge-like when added. During the fattening process, the proportion of water in the fattening substrate decreases. As a result, the consistency of the fattening substrate changes and it becomes free-flowing and portionable.

Similarly, the first, second, and third temperature reference values at time t1 may be different reference values or an identical reference value. The respective temperature reference values at time t1 are preferably obtained from repeated observations of temperature measurement values of the fattening substrate at time t1 and are stored in the processing unit or can be retrieved therefrom.

In a second sub-step, a reference value undershoot is preferably determined at time t1 in the event that one or more of the detected measurement values falls below the respective reference value. In the event that one or more of the detected measurement values exceeds the respective reference value, a reference value overshoot is preferably determined in a third sub-step at time t1.

It is preferably recognised that at one or more of the humidity and/or temperature measuring points, at which a reference value undershoot or a correlation between a reference value and a detected measurement value is in a range between +/−2%, preferably +/−5%, more preferably +/−10%, there is no cluster formation of the insect larvae at time t1.

In a fourth preferred sub-step, a cluster formation of the insect larvae at time t1 is determined at one or more of the humidity and/or temperature measuring points in the event that a reference value overshoot was detected at said one or more humidity and/or temperature measuring points at time t1.

If the reference value is exceeded at one or more of the humidity and/or temperature measuring points, it can be assumed that the measurement value has been falsified due to cluster formation of the insect larvae at one or more of the humidity and/or temperature measuring points. This means that not only the humidity or temperature of the fattening substrate is detected, but also the increasing humidity or temperature of the grouping insect larvae. If the insect larvae group together at a measuring point, this influences the measurement value. In comparison, a measurement value detected at a measuring point where no insect larvae are grouped together is not additionally influenced. For this reason, a cluster formation can be determined at the corresponding points. The measurement values are used to locate the insect larvae at time t1 inside the insect fattening container. Visual observation of the insect larvae to assess the positioning of the insect larvae within the insect fattening container is not necessary.

In a preferred fifth sub-step, the determined cluster formation of the insect larvae at time t1 is compared with a reference cluster formation of the insect larvae at time t1. In the event that the determined cluster formation of the insect larvae at time t1 corresponds to the reference cluster formation of the insect larvae at time t1, a regular activity of the insect larvae at time t1 is preferably determined in a sixth sub-step. However, if the determined cluster formation of the insect larvae at time t1 deviates from the reference cluster formation of the insect larvae at time t1, an irregular activity of the insect larvae at time t1 is preferably determined in a seventh sub-step. Finally, preferably in an eighth sub-step, a development status signal is output at time t1 depending on the determined activity of the insect larvae at time t1.

The reference cluster formation at time t1 is preferably determined from repeated observations of cluster formations or distribution patterns of the insect larvae during the fattening process at time t1 and is stored in the processing unit or can be retrieved thereby. Observations have shown that at the beginning of a fattening phase, the insect larvae are essentially evenly distributed within the insect fattening container. With increasing activity, the insect larvae initially group together in a central portion of the insect fattening container and then also at the edges or corners of the insect fattening container.

A correlation between a determined cluster formation and a reference cluster formation exists if it is known from the observations that at said one or more of the humidity and/or temperature measuring points, at which a cluster formation of the insect larvae was recognised, a cluster formation is known to be present at time t1. The insect larvae therefore exhibit regular activity, as is known from repeated observations.

A deviation between a determined cluster formation and a reference cluster formation exists in particular if it is known from observations that at said one or more of the humidity and/or temperature measuring points, at which a cluster formation of the insect larvae was recognised, there is usually no cluster formation at the time t1. As a result, an irregular activity of the insect larvae is determined. This may indicate an illness, an inadequate climate, or poor nutrition. This enables appropriate countermeasures to be taken.

The method preferably further comprises processing the detected first, second, and/or third measured humidity values and/or the detected first, second, and/or third measured temperature values by means of the processing unit at a second time t2, at a third time t3, at a fourth time t4, at a fifth time t5, at a sixth time t6, and/or at a seventh time t7. In principle, any number of time points may be provided for in the process, so that a number of one, two, three, four, five, six, seven, eight, nine, ten, eleven, twelve or any number of others may be provided. The measured values are used to locate the insects inside the insect fattening container at different times. As a result, the movement of the insect larvae within the insect fattening container can be tracked over a certain period of time.

Between the time t1 and the time t2 there is preferably a period of between one hour and 48 hours, preferably between 12 hours and 24 hours, particularly preferably around 24 hours. Preferably, there is approximately the same amount of time between the points in time in order to increase comparability.

Between the time t2 and the time t3 there is preferably also a period of between one hour and 48 hours, preferably between 12 hours and 24 hours, particularly preferably around 24 hours.

Between time t3 and time t4 there is preferably also a period of between one hour and 48 hours, preferably from 12 hours to 24 hours, particularly preferably about 24 hours.

Between time t4 and time t5 there is preferably also a period of between one hour and 48 hours, preferably from 12 hours to 24 hours, particularly preferably about 24 hours, and preferably between time t5 and time t6 a period of between one hour and 48 hours, preferably from 12 hours to 24 hours, particularly preferably 24 hours.

Between time t6 and time t7 there is also preferably a period of between one hour and 48 hours, preferably from 12 hours to 24 hours, particularly preferably from around 24 hours.

The processing of the recorded measured values at time t2 comprises several sub-steps. In a first sub-step, the detected first measured humidity value at the time t2 is first compared with a first humidity reference value at the time t2, the detected second measured humidity value at the time t2 is compared with a second humidity limit value at the time t2, the detected third measured humidity value at the time t2 is compared with a third humidity reference value at the time t2, the detected first measured temperature value at the time t2 is compared with a first temperature reference value at the time t2, the detected second measured temperature value at the time t2 is compared with a second temperature reference value at the time t2, and the detected third measured temperature value at the time t2 is compared with a third temperature reference value at the time t2.

The first, second, and third humidity reference values at time t2 may be different reference values or an identical reference value. The respective humidity reference values at the time t2 are preferably determined from repeated observations of measured humidity values of the fattening substrate at the time t2 and stored in the processing unit or can be retrieved from it.

Similarly, the first, second, and third temperature reference values at time t2 may be different reference values or an identical reference value. The respective temperature reference values at the time t2 are preferably determined from repeated observations of measured temperature values of the fattening substrate at the time t2 and stored in the processing unit or can be retrieved from it.

In a second sub-step, a reference value undershoot is preferably determined at time t2 in the event that one or more of the recorded measured values falls below the respective reference value. In the event that one or more of the recorded measured values exceed the respective reference value, a reference value overshoot is preferably determined in a third sub-step at the time t2.

It is preferably recognised that at one or more of the humidity and/or temperature measuring points, at which a reference value undershoot or an agreement between a reference value and a recorded measured value is in a range between +/−2%, preferably +/−5%, more preferably +/−10%, there is no clustering of the insect larvae at the time t2.

In a fourth preferred sub-step, a clustering of the insect larvae at the time t2 is determined at one or more of the humidity and/or temperature measurement points in the event that a reference value overshoot was detected at said one or more humidity and/or temperature measurement points at the time t2.

If the reference value is exceeded at one or more of the humidity and/or temperature measuring points at time t2, it can be assumed that the measured value was falsified due to clustering of the insect larvae at one or more of the humidity and/or temperature measuring points. This means that at time t2, not only the humidity or temperature of the fattening substrate is recorded, but also the increasing humidity or temperature of the grouping insect larvae. The measured values are used to locate the insect larvae at time t2 inside the insect fattening container.

In a preferred fifth sub-step, the determined cluster formation of the insect larvae at time t2 is compared with a reference cluster formation of the insect larvae at time t2. In the event that the determined cluster formation of the insect larvae at time t2 corresponds to the reference cluster formation of the insect larvae at time t2, a regular activity of the insect larvae at time t2 is preferably determined in a sixth sub-step. However, if the determined cluster formation of the insect larvae at time t2 deviates from the reference cluster formation of the insect larvae at time t2, an irregular activity of the insect larvae at time t2 is preferably determined in a seventh sub-step. Finally, preferably in an eighth sub-step, a development status signal is output at time t2 as a function of the determined activity of the insect larvae at time t2.

The reference cluster formation at time t2 is preferably determined from repeated observations of cluster formations or distribution patterns of the insect larvae during the fattening process at time t2 and is stored in the processing unit or can be retrieved by it.

A match between a determined cluster formation and a reference cluster formation exists if it is known from the observations that at said one or more of the humidity and/or temperature measuring points, at which a cluster formation of the insect larvae was recognised, a cluster formation is known to be present at the time t2. The insect larvae therefore exhibit regular activity at time t2, as is known from repeated observations.

A deviation between a determined cluster formation and a reference cluster formation exists in particular if it is known from observations that at said one or more of the humidity and/or temperature measuring points, at which a cluster formation of the insect larvae was recognised, there is usually no cluster formation at the time t2. As a result, an irregular activity of the insect larvae is determined at time t2.

The processing of the recorded measured values at time t3 preferably also comprises several sub-steps. The sub-steps are preferably analogue to the sub-steps that were carried out as part of the processing of the recorded measured values at time t1 and time t2. In a first sub-step, preferably first the detected first humidity measured value at the time t3 is compared with a first humidity reference value at the time t3, the detected second measured humidity value at the time t3 is compared with a second humidity limit value at the time t3, the detected third measured humidity value at the time t3 is compared with a third humidity reference value at the time t3, the detected first measured temperature value at the time t3 is compared with a first temperature reference value at the time t3, the detected second measured temperature value at the time t3 is compared with a second temperature reference value at the time t3, and/or the detected third measured temperature value at the time t3 is compared with a third temperature reference value at the time t3.

The first, second, and/or third humidity reference values at time t3 may be different reference values or an identical reference value. The respective humidity reference values at the time t3 are preferably obtained from repeated observations of measured humidity values of the fattening substrate at the time t3 and are stored in the processing unit or can be retrieved by it.

Similarly, the first, second, and/or third temperature reference values at time t3 may be different reference values or an identical reference value. The respective temperature reference values at the time t3 are preferably obtained from repeated observations of measured temperature values of the fattening substrate at the time t3 and are stored in the processing unit or can be retrieved from it.

In a second sub-step, a reference value undershoot is preferably determined at time t3 in the event that one or more of the recorded measured values falls below the respective reference value. In the event that one or more of the recorded measured values exceeds the respective reference value, a reference value overshoot is preferably determined in a third sub-step at the time t3.

It is preferably detected that at one or more of the humidity and/or temperature measuring points, at which a reference value undershoot or an agreement between a reference value and a recorded measured value is in a range between +/−2%, preferably +/−5%, more preferably +/−10%, there is no clustering of the insect larvae at time t3.

In a fourth preferred sub-step, a clustering of the insect larvae at the time t3 at one or more of the humidity and/or temperature measurement points is determined in the event that a reference value overshoot at the time t3 was detected at said one or more humidity and/or temperature measurement points.

If the reference value is exceeded at one or more of the humidity and/or temperature measuring points at time t3, it can be assumed that the measured value was falsified due to clustering of the insect larvae at one or more of the humidity and/or temperature measuring points. This means that at time t3, not only the humidity or temperature of the fattening substrate is recorded, but also the increasing humidity or temperature of the grouping insect larvae. The measured values are used to locate the insect larvae at time t3 inside the insect fattening container.

In a preferred fifth sub-step, the determined clustering of the insect larvae at time t3 is compared with a reference clustering of the insect larvae at time t3. In the event that the determined cluster formation of the insect larvae at time t3 corresponds to the reference cluster formation of the insect larvae at time t3, a regular activity of the insect larvae at time t3 is preferably determined in a sixth sub-step. However, if the determined cluster formation of the insect larvae at time t3 deviates from the reference cluster formation of the insect larvae at time t3, an irregular activity of the insect larvae at time t3 is preferably determined in a seventh sub-step. Finally, preferably in an eighth sub-step, a development status signal is output at time t3 as a function of the determined activity of the insect larvae at time t3.

The reference cluster formation at time t3 is preferably determined from repeated observations of cluster formations or distribution patterns of the insect larvae during the fattening process at time t3 and is stored in the processing unit or can be retrieved by it.

A match between a determined cluster formation and a reference cluster formation exists if it is known from the observations that at these one or more of the humidity and/or temperature measuring points, at which a cluster formation of the insect larvae was recognised, a cluster formation is known to be present at the time t3. The insect larvae therefore exhibit regular activity at time t3, as is known from repeated observations.

A deviation between a determined cluster formation and a reference cluster formation exists in particular if it is known from observations that at said one or more of the humidity and/or temperature measuring points, at which a cluster formation of the insect larvae was recognised, there is usually no cluster formation at the time t3. As a result, an irregular activity of the insect larvae is determined at time t3.

The processing of the recorded measured values at time t4 preferably also comprises several sub-steps. The sub-steps are preferably analogue to the sub-steps that were carried out as part of the processing of the recorded measured values at time t1, time t2, and time t3. In a first sub-step, preferably first the detected first humidity measured value at the time t4 is compared with a first humidity reference value at the time t4, the detected second measured humidity value at the time t4 is compared with a second humidity limit value at the time t4, the detected third measured humidity value at the time t4 is compared with a third humidity reference value at the time t4, the detected first measured temperature value at the time t4 is compared with a first temperature reference value at the time t4, the detected second measured temperature value at the time t4 is compared with a second temperature reference value at the time t4, and/or the detected third measured temperature value at the time t4 is compared with a third temperature reference value at the time t4.

The first, second, and/or third humidity reference values at time t4 may be different reference values or an identical reference value. The respective humidity reference values at the time t4 are preferably obtained from repeated observations of measured humidity values of the fattening substrate at the time t4 and are stored in the processing unit or can be retrieved by it.

Similarly, the first, second, and/or third temperature reference values at time t4 may be different reference values or an identical reference value. The respective temperature reference values at the time t4 are preferably obtained from repeated observations of measured temperature values of the fattening substrate at the time t4 and are stored in the processing unit or can be retrieved from it.

In a second sub-step, a reference value undershoot is preferably determined at time t4 in the event that one or more of the recorded measured values falls below the respective reference value. In the event that one or more of the recorded measured values exceeds the respective reference value, a reference value overshoot is preferably determined in a third sub-step at the time t4.

It is preferably recognised that at one or more of the humidity and/or temperature measuring points, at which a reference value undershoot or an agreement between a reference value and a recorded measured value is in a range between +/−2%, preferably +/−5%, more preferably +/−10%, there is no clustering of the insect larvae at the time t4.

In a fourth preferred sub-step, a clustering of the insect larvae at the time t4 at one or more of the humidity and/or temperature measurement points is determined in the event that a reference value overshoot at the time t4 was detected at said one or more humidity and/or temperature measurement points. The measured values are used to locate the insect larvae at time t4 inside the insect fattening container.

If the reference value is exceeded at one or more of the humidity and/or temperature measuring points at time t4, it can be assumed that the measured value was falsified due to clustering of the insect larvae at one or more of the humidity and/or temperature measuring points. This means that at time t4, not only the humidity or temperature of the fattening substrate is recorded, but also the increasing humidity or temperature of the grouping insect larvae.

In a preferred fifth sub-step, the determined clustering of the insect larvae at time t4 is compared with a reference clustering of the insect larvae at time t4. In the event that the determined cluster formation of the insect larvae at time t4 corresponds to the reference cluster formation of the insect larvae at time t4, a regular activity of the insect larvae at time t4 is preferably determined in a sixth sub-step. However, if the determined cluster formation of the insect larvae at time t4 deviates from the reference cluster formation of the insect larvae at time t4, an irregular activity of the insect larvae at time t4 is preferably determined in a seventh sub-step. Finally, preferably in an eighth sub-step, a development status signal is output at time t4 as a function of the determined activity of the insect larvae at time t4.

The reference cluster formation at time t4 is preferably determined from repeated observations of cluster formations or distribution patterns of the insect larvae during the fattening process at time t4 and is stored in the processing unit or can be retrieved by it.

A match between a determined cluster formation and a reference cluster formation exists if it is known from the observations that at said one or more of the humidity and/or temperature measuring points, at which a cluster formation of the insect larvae was recognised, a cluster formation is known to be present at the time t4. The insect larvae therefore exhibit regular activity at time t4, as is known from repeated observations.

A deviation between a determined cluster formation and a reference cluster formation exists in particular if it is known from observations that at said one or more of the humidity and/or temperature measuring points, at which a cluster formation of the insect larvae was recognised, there is usually no cluster formation at the time t4. As a result, an irregular activity of the insect larvae is determined at time t4.

The processing of the recorded measured values at time t5 preferably also comprises several sub-steps. The sub-steps are preferably analogue to the sub-steps that were carried out as part of the processing of the recorded measured values at time t1, time t2, time t3 and time t4. In a first sub-step, preferably first the detected first measured humidity value at the time t5 is compared with a first humidity reference value at the time t5, the detected second measured humidity value at the time t5 is compared with a second humidity limit value at the time t5, the detected third measured humidity value at the time t5 is compared with a third humidity reference value at the time t5, the detected first temperature measurement value at the time t5 is compared with a first temperature reference value at the time t5, the detected second temperature measurement value at the time t5 is compared with a second temperature reference value at the time t5, and/or the detected third temperature measurement value at the time t5 is compared with a third temperature reference value at the time t5.

The first, second, and/or third humidity reference values at time t5 may be different reference values or an identical reference value. The respective humidity reference values at the time t5 are preferably obtained from repeated observations of measured humidity values of the fattening substrate at the time t5 and are stored in the processing unit or can be retrieved by it.

Similarly, the first, second, and/or third temperature reference values at time t5 may be different reference values or an identical reference value. The respective temperature reference values at the time t5 are preferably obtained from repeated observations of measured temperature values of the fattening substrate at the time t5 and are stored in the processing unit or can be retrieved by it.

In a second sub-step, a reference value undershoot is preferably determined at the time t5 in the event that one or more of the recorded measured values falls below the respective reference value. In the event that one or more of the recorded measured values exceeds the respective reference value, a reference value overshoot is preferably determined in a third sub-step at the time t5.

It is preferably recognised that at one or more of the humidity and/or temperature measuring points at which a reference value is undershot or a match between a reference value and a recorded measured value is in a range between +/−2%, preferably +/−5%, more preferably +/−10%, there is no clustering of the insect larvae at time t5.

In a fourth preferred sub-step, a clustering of the insect larvae at the time t5 at one or more of the humidity and/or temperature measurement points is determined in the event that a reference value overshoot was detected at said one or more humidity and/or temperature measurement points at the time t5.

If the reference value is exceeded at one or more of the humidity and/or temperature measuring points at time t5, it can be assumed that the measured value was falsified due to clustering of the insect larvae at one or more of the humidity and/or temperature measuring points. This means that at time t5, not only the humidity or temperature of the fattening substrate is recorded, but also the increasing humidity or temperature of the grouping insect larvae. The measured values are used to locate the insect larvae at time t5 inside the insect fattening container.

In a preferred fifth sub-step, the determined cluster formation of the insect larvae at time t5 is compared with a reference cluster formation of the insect larvae at time t5. In the event that the determined cluster formation of the insect larvae at time t5 corresponds to the reference cluster formation of the insect larvae at time t5, a regular activity of the insect larvae at time t5 is preferably determined in a sixth sub-step. However, if the determined cluster formation of the insect larvae at time t5 deviates from the reference cluster formation of the insect larvae at time t5, an irregular activity of the insect larvae at time t5 is preferably determined in a seventh sub-step. Finally, preferably in an eighth sub-step, a development status signal is output at time t5 as a function of the determined activity of the insect larvae at time t5.

The reference cluster formation at time t5 is preferably determined from repeated observations of cluster formations or distribution patterns of the insect larvae during the fattening process at time t5 and is stored in the processing unit or can be retrieved by it.

A match between a determined cluster formation and a reference cluster formation exists if it is known from the observations that at said one or more of the humidity and/or temperature measuring points, at which a cluster formation of the insect larvae was recognised, a cluster formation is known to be present at the time t5. The insect larvae therefore exhibit regular activity at time t5, as is known from repeated observations.

A deviation between a determined cluster formation and a reference cluster formation exists in particular if it is known from observations that at said one or more of the humidity and/or temperature measuring points, at which a cluster formation of the insect larvae was recognised, there is usually no cluster formation at the time t5. As a result, an irregular activity of the insect larvae is determined at time t5.

The processing of the recorded measured values at time t6 preferably also comprises several sub-steps. The sub-steps are preferably analogue to the sub-steps that were carried out as part of the processing of the recorded measured values at time t1, time t2, time t3, time t4 and time t5. In a first sub-step, preferably first the detected first humidity measured value at the time t6 is compared with a first humidity reference value at the time t6, the detected second measured humidity value at the time t6 is compared with a second humidity limit value at the time t6, the detected third measured humidity value at the time t6 is compared with a third humidity reference value at the time t6, the detected first measured temperature value at the time t6 is compared with a first temperature reference value at the time t6, the detected second measured temperature value at the time t6 is compared with a second temperature reference value at the time t6, and/or the detected third measured temperature value at the time t6 is compared with a third temperature reference value at the time t6.

The first, second, and/or third humidity reference values at time t6 may be different reference values or an identical reference value. The respective humidity reference values at the time t6 are preferably obtained from repeated observations of measured humidity values of the fattening substrate at the time t6 and are stored in the processing unit or can be retrieved by it.

Similarly, the first, second, and/or third temperature reference values at time t6 may be different reference values or an identical reference value. The respective temperature reference values at the time t6 are preferably obtained from repeated observations of measured temperature values of the fattening substrate at the time t6 and are stored in the processing unit or can be retrieved by it.

In a second sub-step, a reference value undershoot is preferably determined at time t6 in the event that one or more of the recorded measured values falls below the respective reference value. In the event that one or more of the recorded measured values exceeds the respective reference value, a reference value overshoot is preferably determined in a third sub-step at the time t6.

It is preferably recognised that at one or more of the humidity and/or temperature measuring points at which a reference value is not reached or a match between a reference value and a recorded measured value is in a range between +/−2%, preferably +/−5%, more preferably +/−10%, there is no clustering of the insect larvae at time t6.

In a fourth preferred sub-step, a clustering of the insect larvae at the time t6 at one or more of the humidity and/or temperature measurement points is determined in the event that a reference value overshoot at the time t6 was detected at said one or more of the humidity and/or temperature measurement points.

If the reference value is exceeded at one or more of the humidity and/or temperature measuring points at time t6, it can be assumed that the measured value was falsified due to clustering of the insect larvae at one or more of the humidity and/or temperature measuring points. This means that at time t6, not only the humidity or temperature of the fattening substrate is recorded, but also the increasing humidity or temperature of the grouping insect larvae. The measured values are used to locate the insect larvae at time t6 inside the insect fattening container.

In a preferred fifth sub-step, the determined clustering of the insect larvae at time t6 is compared with a reference clustering of the insect larvae at time t6. In the event that the determined cluster formation of the insect larvae at time t6 corresponds to the reference cluster formation of the insect larvae at time t6, a regular activity of the insect larvae at time t6 is preferably determined in a sixth sub-step. However, if the determined cluster formation of the insect larvae at time t6 deviates from the reference cluster formation of the insect larvae at time t6, an irregular activity of the insect larvae at time t6 is preferably determined in a seventh sub-step. Finally, preferably in an eighth sub-step, a development status signal is output at time t6 as a function of the determined activity of the insect larvae at time t6.

The reference cluster formation at time t6 is preferably determined from repeated observations of cluster formations or distribution patterns of the insect larvae during the fattening process at time t6 and is stored in the processing unit or can be retrieved by it.

A match between a determined cluster formation and a reference cluster formation exists if it is known from the observations that at said one or more of the humidity and/or temperature measuring points, at which a cluster formation of the insect larvae was recognised, a cluster formation is known from experience to be present at the time t6. The insect larvae therefore exhibit regular activity at time t6, as is known from repeated observations.

A deviation between a determined cluster formation and a reference cluster formation exists in particular if it is known from observations that at said one or more of the humidity and/or temperature measuring points, at which a cluster formation of the insect larvae was recognised, there is usually no cluster formation at the time t6. As a result, an irregular activity of the insect larvae is determined at time t6.

The processing of the recorded measured values at time t7 preferably also comprises several sub-steps. The sub-steps are preferably analogue to the sub-steps that were carried out as part of the processing of the recorded measured values at time t1, time t2, time t3, time t4, time t5, and time t6. In a first sub-step, preferably first the recorded first measured humidity value at the time t7 is compared with a first humidity reference value at the time t7, the recorded second measured humidity value at the time t7 is compared with a second humidity limit value at the time t7, the recorded third measured humidity value at the time t7 is compared with a third humidity reference value at the time t7, the detected first temperature measurement value at the time t7 is compared with a first temperature reference value at the time t7, the detected second temperature measurement value at the time t7 is compared with a second temperature reference value at the time t7, and/or the detected third temperature measurement value at the time t7 is compared with a third temperature reference value at the time t7.

The first, second, and/or third humidity reference values at time t7 may be different reference values or an identical reference value. The respective humidity reference values at the time t7 are preferably obtained from repeated observations of measured humidity values of the fattening substrate at the time t7 and are stored in the processing unit or can be retrieved by it.

Similarly, the first, second, and/or third temperature reference values at time t7 may be different reference values or an identical reference value. The respective temperature reference values at the time t7 are preferably obtained from repeated observations of measured temperature values of the fattening substrate at the time t7 and are stored in the processing unit or can be retrieved from it.

In a second sub-step, a reference value undershoot is preferably determined at the time t7 in the event that one or more of the recorded measured values falls below the respective reference value. In the event that one or more of the recorded measured values exceeds the respective reference value, a reference value overshoot is preferably determined in a third sub-step at the time t7.

It is preferably recognised that at one or more of the humidity and/or temperature measuring points at which a reference value is not reached or a match between a reference value and a recorded measured value is in a range between +/−2%, preferably +/−5%, more preferably +/−10%, there is no clustering of the insect larvae at time t7.

In a fourth preferred sub-step, a clustering of the insect larvae at the time t7 at one or more of the humidity and/or temperature measurement points is determined in the event that a reference value overshoot at the time t7 was detected at said one or more humidity and/or temperature measurement points.

If the reference value is exceeded at one or more of the humidity and/or temperature measuring points at time t7, it can be assumed that the measured value was falsified due to clustering of the insect larvae at one or more of the humidity and/or temperature measuring points. This means that at time t7, not only the humidity or temperature of the fattening substrate is recorded, but also the increasing humidity or temperature of the grouping insect larvae. The measured values are used to locate the insect larvae at time t7 inside the insect fattening container.

In a fifth preferred sub-step, the determined clustering of the insect larvae at time t7 is compared with a reference clustering of the insect larvae at time t7. In the event that the determined cluster formation of the insect larvae at time t7 corresponds to the reference cluster formation of the insect larvae at time t7, a regular activity of the insect larvae at time t7 is preferably determined in a sixth sub-step. However, if the determined cluster formation of the insect larvae at time t7 deviates from the reference cluster formation of the insect larvae at time t7, an irregular activity of the insect larvae at time t7 is preferably determined in a seventh sub-step. Finally, preferably in an eighth sub-step, a development status signal is output at time t7 as a function of the determined activity of the insect larvae at time t7.

The reference cluster formation at time t7 is preferably determined from repeated observations of cluster formations or distribution patterns of the insect larvae during the fattening process at time t7 and is stored in the processing unit or can be retrieved by it.

A match between a determined cluster formation and a reference cluster formation exists if it is known from the observations that at said one or more of the humidity and/or temperature measuring points, at which a cluster formation of the insect larvae was recognised, a cluster formation is known to be present at the time t7. The insect larvae therefore exhibit regular activity at time t7, as is known from repeated observations.

A deviation between a determined cluster formation and a reference cluster formation exists in particular if it is known from observations that at said one or more of the humidity and/or temperature measuring points, at which a cluster formation of the insect larvae was recognised, there is usually no cluster formation at the time t7. As a result, an irregular activity of the insect larvae is determined at time t7.

The method for transporting insect larvae preferably comprises the steps: continuing the fattening phase in the event that the developmental status signal indicates regular activity of the insect larvae, and interrupting the fattening phase in the event that the developmental status signal indicates irregular activity of the insect larvae.

The fattening phase can therefore be interrupted at any of the times t1, t2, t3, t4, 15, 16 or t7 and the user of the insect larvae rearing device can therefore intervene in the fattening process. This makes it possible to intervene in the fattening process at an early stage if irregular activity of the insect larvae is recognised.

If the fattening process is continued without interruption, the insect larvae can be harvested at time t7 or at a defined time after time t7. It is also possible to predict the amount of insect larvae harvested if the measuring points are disposed appropriately, which not only allows the formation of clusters but also the size of the clusters to be determined.

In a preferred refinement, the method further comprises the steps: Determining an average temperature based on the first, second, and/or third measured temperature values at the times t1, t2, t3, t4, t5, t6 and/or t7, determining a substrate evaporation based on the determined average temperatures and a predetermined air humidity, and determining a dry matter content of the fattening substrate based on the determined fattening substrate evaporation.

The estimation of the dry matter content can be used to correct the falsified measured values due to clustering of the insect larvae at one or more of the humidity and/or temperature measuring points.

The present invention also achieves the above object by means of a computer program comprising a program code which, when executed on a processing unit of an insect larvae rearing device, causes the processing unit to execute a method according to one of the above-described preferred embodiments of a method according to the fourth consideration of the invention.

Embodiments of the invention are now be described below with reference to the drawings. Said drawings are not necessarily intended to depict the embodiments to scale, but rather the drawings are schematic and/or slightly distorted as needed for better explanation. With respect to additions to the teachings directly evident from the drawings, reference is made to the relevant prior art. It must be taken into account that various modifications and changes relating to the shape and detail of an embodiment can be made without deviating from the general idea of the invention. The features of the invention disclosed in the description, in the drawings, and in the claims may be essential to the refinement of the invention both individually and in any arbitrary combination. All combinations of two or more of the features disclosed in the description, the drawings, and/or the claims also fall within the scope of the invention. The general idea of the invention is not limited to the precise form or the detail of the preferred embodiments shown and described below or limited to a subject-matter that would be limited in comparison with the subject-matter claimed in the claims. In the case of the specified measurement ranges, values lying within the stated limits should also be disclosed as limit values and be able to be used and claimed as required. For simplicity, the same reference numerals are used below for identical or similar parts, or for parts having identical or similar functions.

Further advantages, features, and details of the invention arise from the following description of the preferred embodiments and from the drawings; said drawings showing in:

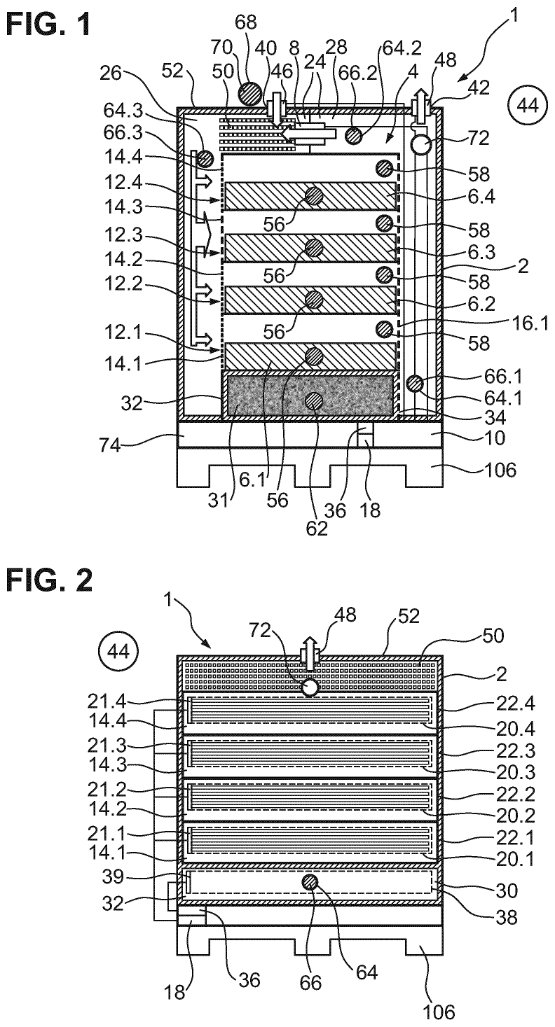

FIG. 1 a section through a first embodiment example of the mobile transport device;

FIG. 2 a further section through the mobile transport device according to FIG. 1, perpendicular to the section of FIG. 1;

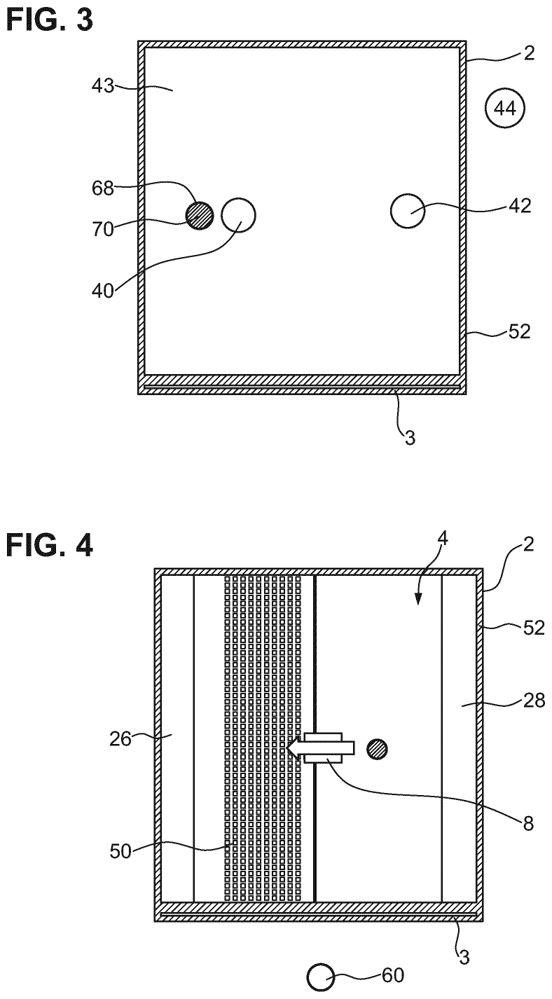

FIG. 3 a top view of the mobile transport device with insulated cover plate of the housing;

FIG. 4 a horizontal section through the mobile transport device;

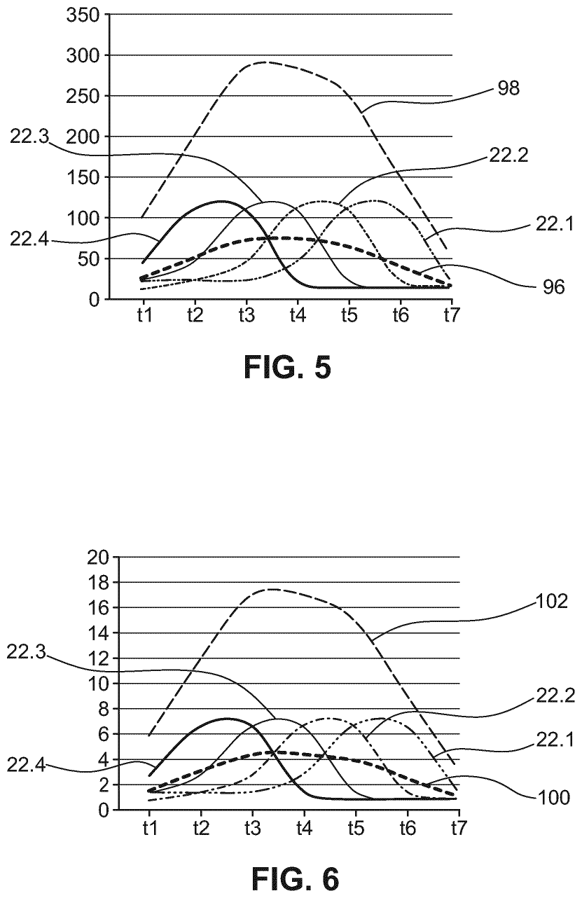

FIG. 5 the heat generation of the compartments over time;

FIG. 6 the ventilation requirements of the compartments over time;

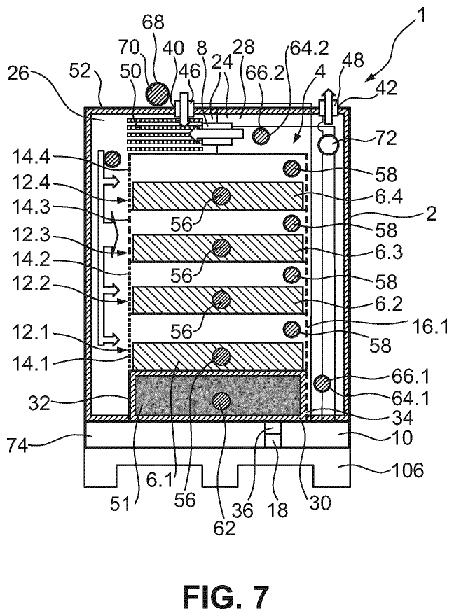

FIG. 7 a section through a second embodiment example of the mobile transport device;

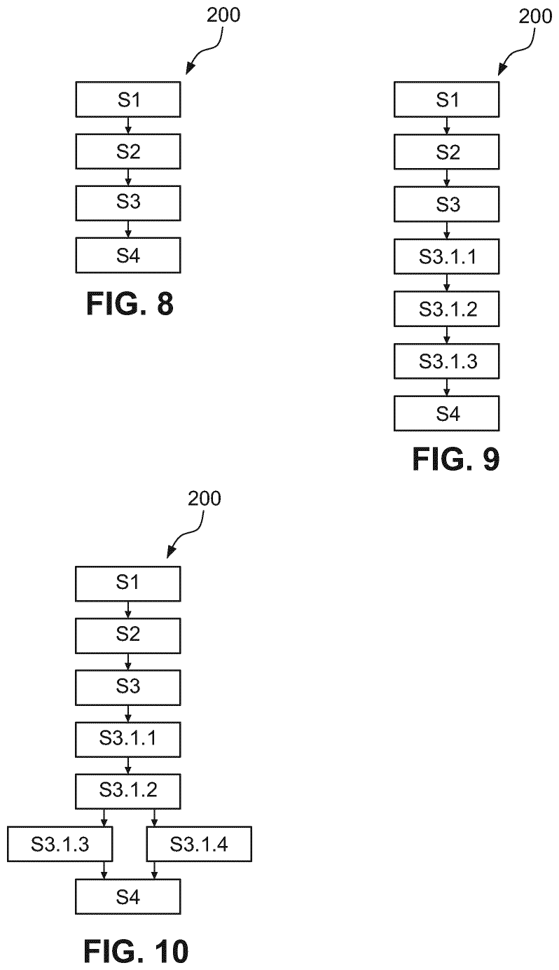

FIG. 8 a schematic flow chart for a first preferred embodiment example of the method for transporting insect larvae;

FIG. 9 a schematic flow chart for a second preferred embodiment example of the method for transporting insect larvae, which is a possible refinement of the first embodiment example of the method for transporting insect larvae;

FIG. 10 a schematic flow chart for a third preferred embodiment example of the method for transporting insect larvae, which is a possible refinement of the second embodiment example of the method for transporting insect larvae;

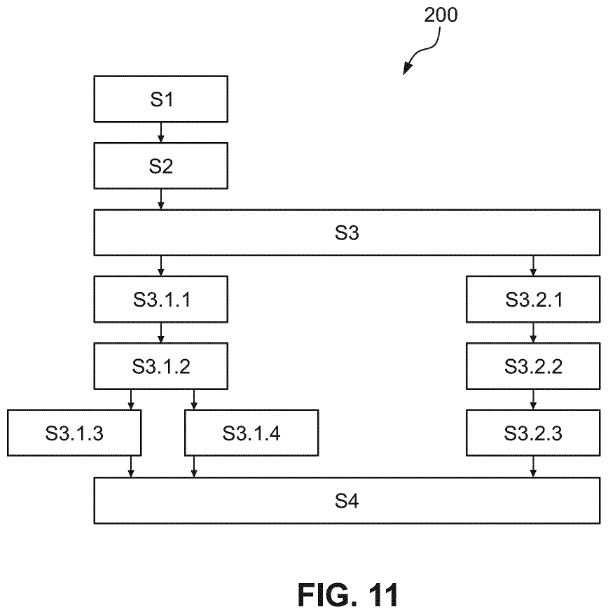

FIG. 11 a schematic flow chart for a fourth preferred embodiment example of the method for transporting insect larvae, which is a possible refinement of the first, second or third embodiment example of the method for transporting insect larvae;

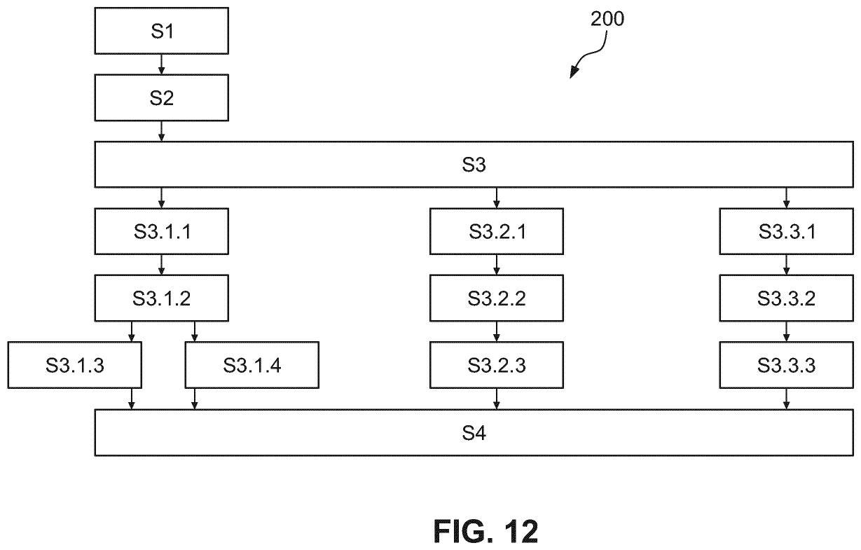

FIG. 12 a schematic flow chart for a fifth preferred embodiment example of the method for transporting insect larvae, which is a possible refinement of the first, second, third or fourth embodiment example of the method for transporting insect larvae;

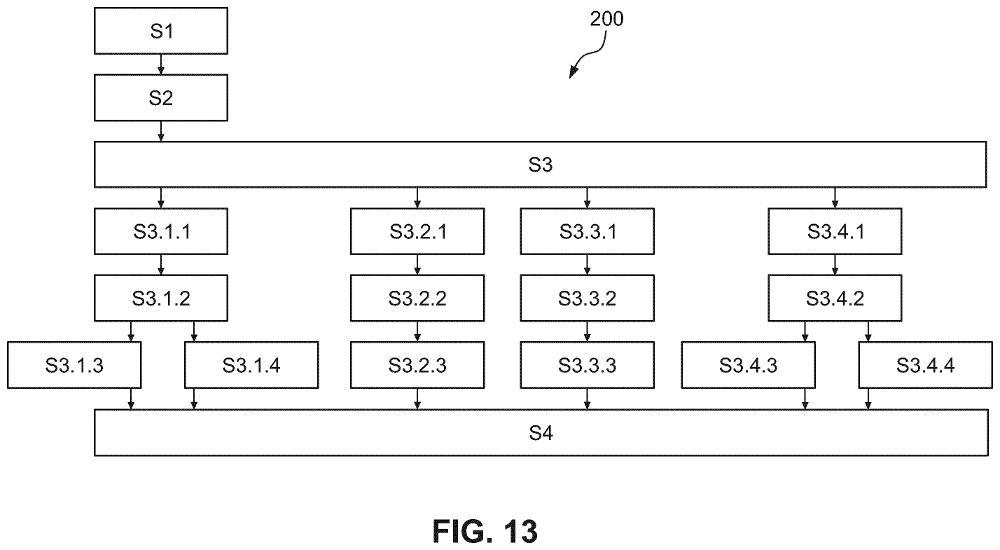

FIG. 13 a schematic flow chart for a sixth preferred embodiment example of the method for transporting insect larvae, which is a possible refinement of the first, second, third, fourth or fifth embodiment example of the method for transporting insect larvae;

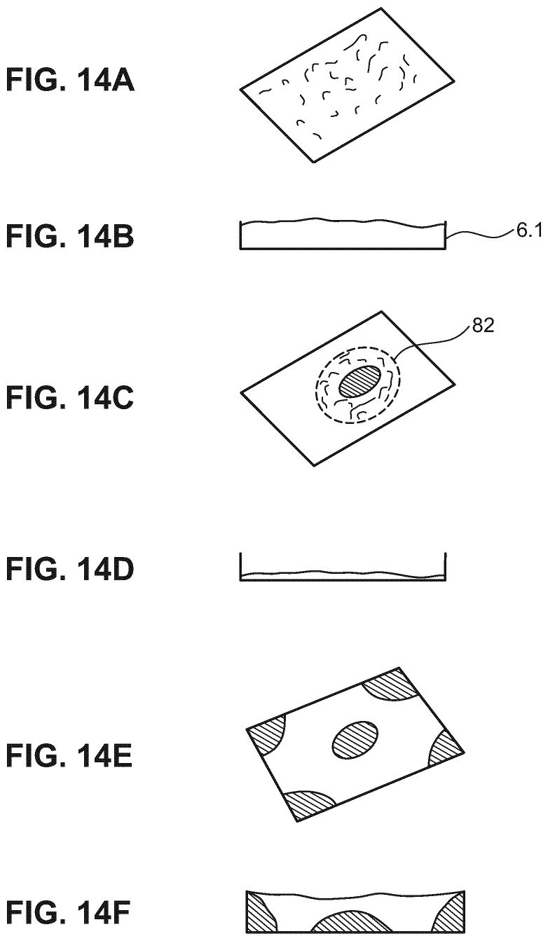

FIG. 14A an isometric top view of a schematic representation of a larval distribution at the beginning of a fattening phase;

FIG. 14B a side view of a schematic representation of the fattening substrate at the beginning of a fattening phase;

FIG. 14C an isometric top view of a schematic representation of a larval distribution in the centre of a fattening phase;

FIG. 14D a side view of a schematic representation of the fattening substrate in the centre of a fattening phase;

FIG. 14E an isometric top view of a schematic representation of a larval distribution at the end of a fattening phase;

FIG. 14F a side view of a schematic representation of a larval distribution at the end of a fattening phase;

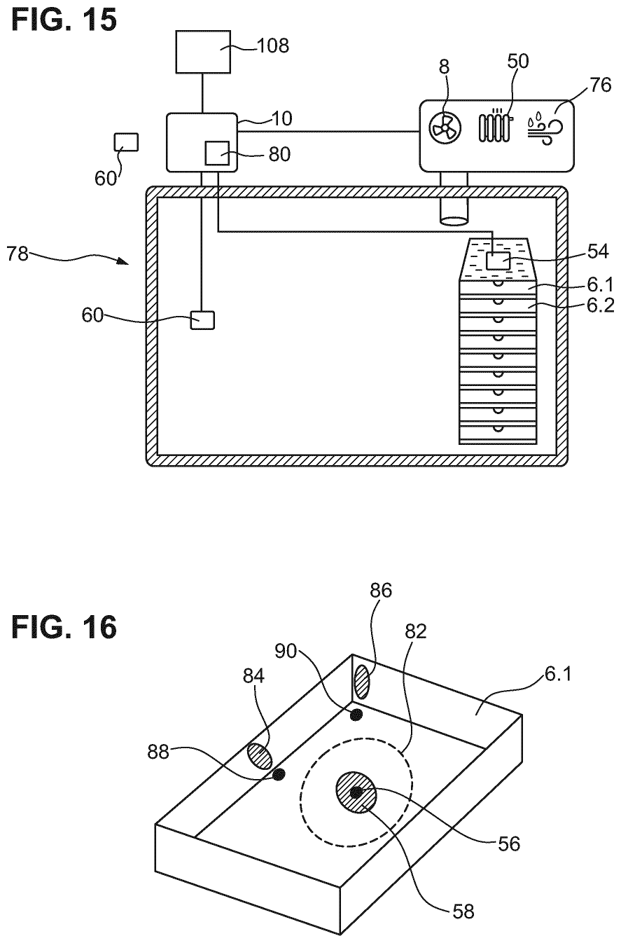

FIG. 15 a schematic view of a stationary insect larvae rearing device;

FIG. 16 an isometric representation of an insect fattening container with activity sensor device for the insect larvae rearing device;

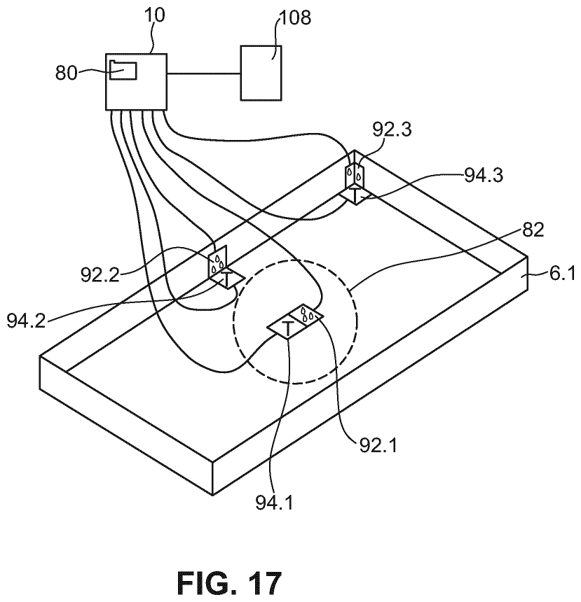

FIG. 17 a further isometric representation of an insect fattening container with activity sensor device for the insect larvae rearing device;

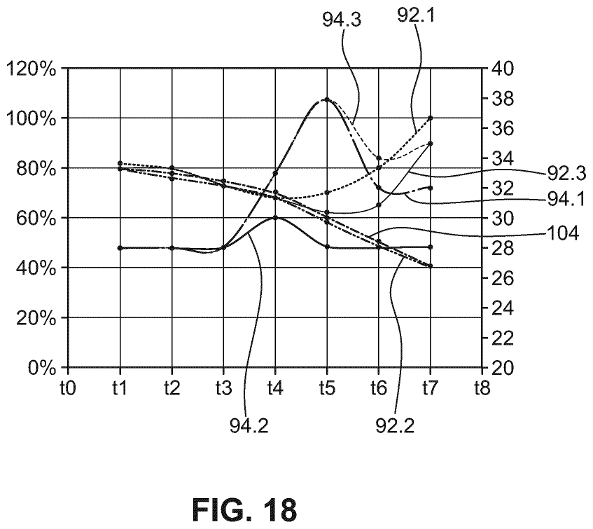

FIG. 18 a time curve of the measured values recorded by the humidity and temperature sensors;

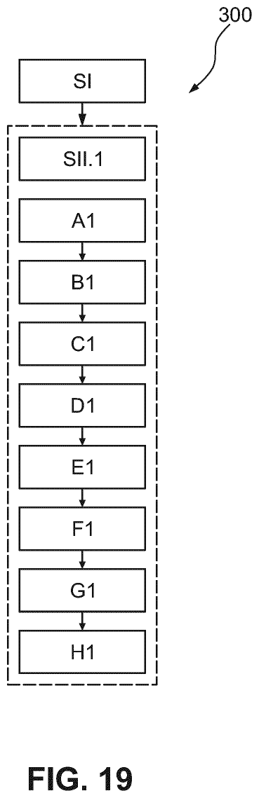

FIG. 19 a schematic flow chart for a first preferred embodiment example of the method for determining the activity of insect larvae;

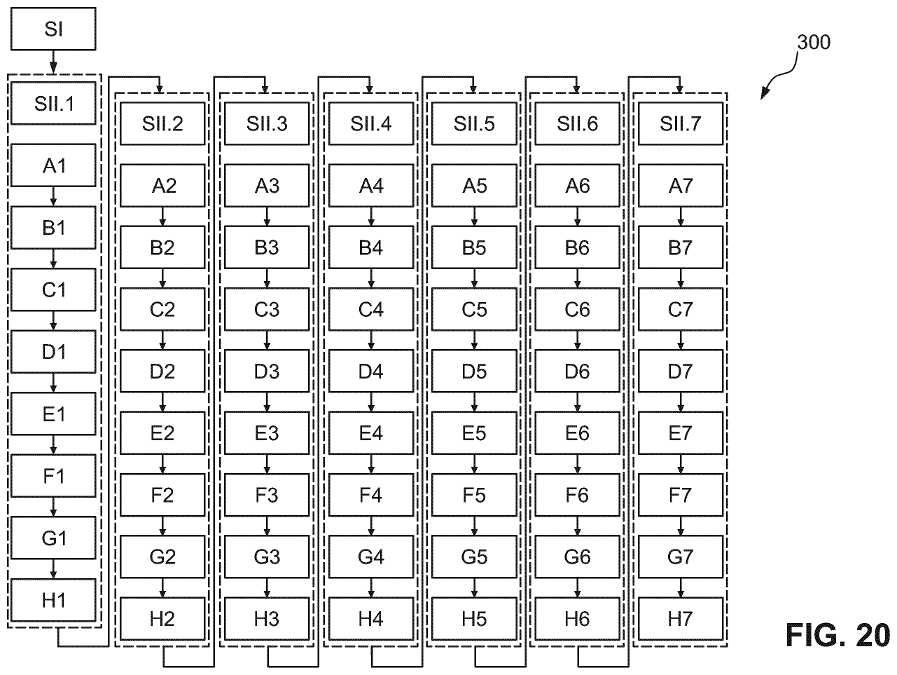

FIG. 20 a schematic flow chart for a second preferred embodiment example of the method for determining an activity of insect larvae, which is a possible refinement of the first embodiment example of the method for determining an activity of insect larvae;

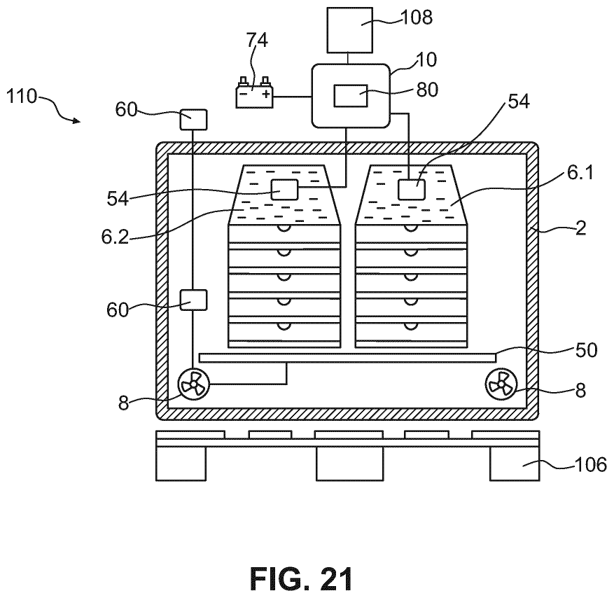

FIG. 21 a second embodiment example of a mobile insect larvae rearing device; and in

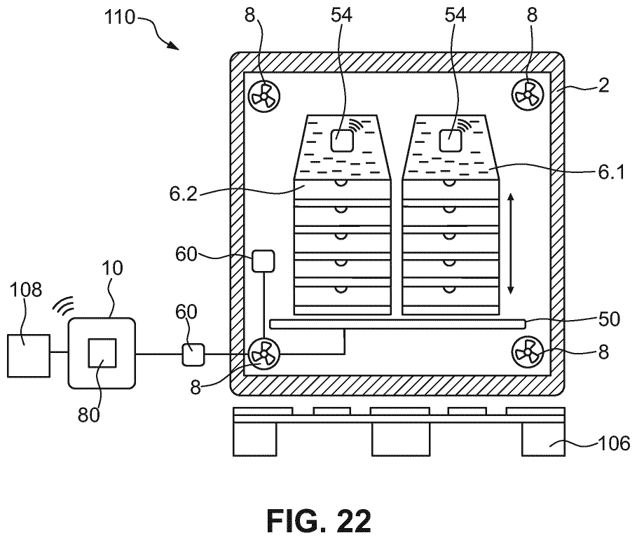

FIG. 22 a third embodiment example of a mobile insect larvae rearing device.

A mobile transport device 1 according to the first consideration has a housing 2 with a thermal insulation 52, an air inlet section 40 and an air outlet section 42 (FIG. 1). Even though the mobile transport device 1 is described here as mobile, i.e. transportable and intended for transport, its functions and features are also useful in stationary devices for rearing and breeding insect larvae, and it should be understood that these functions and features can also be used to advantage in stationary devices. Even if the mobile transport device 1 is initially described without an activity sensor device 54, it should be understood that the mobile transport device may comprise one or more activity sensor devices 54, as will be explained in more detail, in particular with reference to FIGS. 21 and 22.

A receiving section 4 is provided within the housing, in which four insect fattening containers 6.1-6.4 are disposed in the embodiment example of FIG. 1. The receiving section 4 is divided into four compartments 22.1-22.4 (see FIG. 2) for receiving the four insect fattening containers 6.1-6.4, which are disposed vertically and essentially over the entire cross-section of the mobile transport device 1 (see FIGS. 2 and 4). In this embodiment example, the insect fattening containers 6.1-6.4 can be selectively inserted into and removed from the compartments 22.1-22.4. Preferably, the insect fattening containers 6.1-6.4 are filled with insect larvae and fattening substrate before being transported to compartments 22.1-22.4. This can be done manually, for example, by an employee. The fattening area of an insect fattening container 6.1-6.4 is preferably in the range of 0.5 m² to 0.7 m². The fattening substrate, which is added to the insect fattening containers 6.1-6.4 at the beginning, contains a proportion of water. The fattening substrate to be added preferably comprises a proportion of water-binding substances. The fattening substrate to be added preferably comprises a proportion of nutrients. The fattening substrate loses humidity during the fattening process. The insect larvae, the water-binding substances and/or the ventilation/air conditioning remove humidity from the fattening substrate. The consistency of the fattening substrate changes as a result.

After transport, the individual insect fattening containers 6.1-6.4 are then removed from compartments 22.1-22.4. They can then be transported by the recipient to an existing facility at the destination for further rearing and feeding, for example, or harvested directly when ripe. The mobile transport device 1 of the invention allows further feeding and rearing even during transport, which can improve the efficiency of breeding and also the quality of the larvae.

The receiving section 4 divides an interior 24 of the housing 2 into an exhaust section 28 and a ventilation section 26, the function of which will be described in more detail below. The four compartments 22.1-22.4 each have an air regulating device 12.1-12.4, wherein the air regulating devices 12.1-12.4 each have a ventilation section 14.1-14.4 and a exhaust section 16.1-16.4. In the embodiment example of FIG. 1, the ventilation sections 14.1-14.4 each form a first side wall of a compartment and the exhaust sections 16.1-16.4 each form a second side wall of a compartment. The first and second side walls are disposed opposite each other. The ventilation sections 14.1-14.4 also comprise flow cross-sections 20.1-20.4 (see FIG. 2), which are adjustable by means of a ventilation control unit 18 disposed in a lower section of the mobile transport device 1.

A recirculation fan 8 is disposed in an upper section of the mobile transport device 1 inside the housing 2. During operation, the recirculation fan 8 conveys air from the exhaust section 28 into the ventilation section 26 and thus forms an air-conducting connection between the exhaust section 28 and the ventilation section 26. The first, second, third and fourth air regulating devices 12.1-12.4 form a further air-conducting connection between the barking ventilation part 26 and the venting part 28. The recirculation fan 8 is controlled by an electronic control unit 10 disposed in a lower section of the mobile tramming device 1. The recirculation fan 8 is inserted in a partition wall which closes off the entire clear cross-section between an inner wall of the housing 2 and the remaining receiving section 4, so that the ventilation part 26 and the venting part 28 are only connected via the recirculation fan 8 on the one hand and the air regulating devices 12.1-12.4 on the other. This ensures that the air conveyed by the recirculation fan 8 actually reaches the individual insect fattening containers 6.1-6.4 to aerate the insect larvae contained therein.

A storage container 30 is also disposed inside the housing 2, which in this embodiment example is also accommodated in the receiving section 4. In other embodiment examples, it can also be provided at a different location. The storage tank 30 is provided together with the four compartments 22.1-22.4 in a vertical arrangement and forms the lower end of the arrangement. In the embodiment example of FIG. 1, the storage tank 30 comprises an additional thermal insulation 52. An air-conditioning material 31, such as zeolite for air dehumidification, is reversibly accommodated in the storage container 30.

The storage tank 30 has a storage tank ventilation section 32 on a first side and a storage tank exhaust section 34 on a second side opposite the first side. The storage tank ventilation section 32 also comprises a storage tank flow cross-section 38 adjustable by means of a storage tank control unit 36 (see FIG. 2).

The storage tank flow cross-section 38 is completely closed in the embodiment example of FIG. 1 and FIG. 2, so that the air from the ventilation section 26 cannot enter the storage tank 30. If it is determined that the humidity of the air in the interior is too high, the storage tank ventilation section 32 can be partially or fully opened so that air can also circulate through the storage tank 30 and thus reduce the humidity of the air. Instead of zeolite as an air-conditioning material 31, other materials are also conceivable, e.g. a cooling material, so that a temperature of the air can be influenced by corresponding actuation of the storage tank ventilation section 32 and the recirculation fan 8.

Like the electronic control unit 10 and the ventilation control unit 18, the storage container control unit 36 is disposed in a lower section of the mobile transport device 1. In the embodiment example of FIG. 1, the storage container control unit 36 and the ventilation control unit 18 are provided as separate control units. In other embodiments, these can also be partially or fully integrated into a single electronic control unit, which then performs the function of some or all of the control units. The lower section also contains an energy storage unit 74 for supplying the electrical and electronic components of the mobile transport device 1. The energy store 74 is preferably designed as a rechargeable battery and preferably has a capacity such that electrical and electronic components can be supplied with electrical energy for the entire duration of the transport. It is preferable that the mobile transport device 1 has an electrical connection (not shown) via which the mobile transport device 1 can be connected to a local power supply. The mobile transport device 1 can therefore also be operated in stationary mode without the energy storage unit 74 providing additional energy.

A fresh air fan 46 is disposed in the air inlet section 40 of the housing 2, which ventilates air from an environment 44 into the interior 24. In the embodiment example of FIG. 1, the air inlet section 40 opens into the ventilation section 26 of the interior 24, so that the air from the environment 44 is ventilated into the ventilation section 26. A heating device 50 is disposed in the ventilation section 26, which heats the incoming air. The heating device 50 is also disposed in such a way that the air recirculated by the recirculation fan 8 can be heated at the same time.

An exhaust fan 48 is disposed in the air outlet section 42 of the housing 2, which directs air from the exhaust section 28 of the interior 24 into the environment 44. Both the fresh air fan 46 and the exhaust air fan 48 can be controlled by the electronic control unit 10.

In the embodiment example of FIG. 1, a first insect fattening container temperature measuring point 56 is disposed in each of the four insect fattening containers 6.1-6.4. In each of the compartments 22.1-22.4, in which the insect fattening containers 6.1-6.4 are accommodated, a first insect fattening container humidity measuring point 58 is also provided, which is, as it were, a further insect fattening container temperature measuring point.

A storage tank temperature measuring point 62 is disposed in the storage tank 30. A first indoor humidity measuring point 64.1 and a first indoor temperature measuring point 66.1 adjacent to the storage tank exhaust section 34, as well as a second indoor humidity measuring point 64.2 and a second indoor temperature measuring point 66.2 adjacent to the recirculation fan 8 are provided in the exhaust section 28. A CO2 measuring point 72, which is likewise a further interior temperature measuring point, is disposed in the exhaust section 28 adjacent to the exhaust fan 48.

A third interior temperature measuring point 66.3 and a third interior humidity measuring point 64.3 are disposed in the ventilation section 26. An outdoor humidity measuring point 68 and an outdoor temperature measuring point 70 are disposed outside the housing 2 in the surroundings 44.

All measuring points are connected to the electronic control unit so that it can analyse the corresponding measuring signals from the measuring points.

The mobile transport device 1 is positioned on a pallet 106. This simplifies transport and the mobile transport device 1 can be handled and transported using conventional logistics equipment.

FIG. 2 shows a side view of a further section of the mobile transport device 1, so that the ventilation sections 14.1-14.4 together with the flow cross-sections 20.1-20.4 and the storage container ventilation section together with the storage container flow cross-section 38 can be seen. In the embodiment example of FIG. 2, the flow cross-sections 20.1-20.4 and the storage tank flow cross-section 38 comprise discs that can be moved by means of an actuator 21.1-21.4 or a storage tank actuator 39. In the embodiment example of FIG. 2, the actuators 21.1-21.4 are controlled by the ventilation control unit 18 and the storage tank actuator 39 is controlled by the storage tank control unit 36.

The storage tank flow cross-section 38 is completely closed. The first, second, and fourth flow cross-sections 20.1, 20.2, 20.4 are partially open so that air from the ventilation section 26 can flow partially into the insect fattening containers 6.1, 6.2, 6.4. The third flow cross-section 20.3, on the other hand, is completely open so that the air can flow into the third insect fattening container 6.3 via the completely open flow cross-section 20.3. As also indicated in FIG. 1 by the arrow in the ventilation section 26, a roughly equal air flow enters the first, second, and fourth compartments 22.1, 22.2, 22.4, and a slightly higher proportion enters the third compartment 22.3. During the breeding phase, the amount of heat produced by the larvae changes, as will be described in more detail. It is typically low at the beginning and then increases after a few days, only to decrease again towards the end of the ripening process. This can be explained in particular by frictional heat caused by the larvae rubbing against each other. As individual compartments 22.1-22.4 can be individually ventilated, the corresponding insect larvae cohort present in the respective compartment 22.1-22.4 can be supplied with an individual and, depending on maturity, adequate air flow in order to be able to optimally adjust the climate in each case.

FIG. 3 shows a top view of the mobile transport device 1 with insulated cover plate 43, which is part of the housing 2. A selectively openable and closable opening 3 is closed in FIG. 3. The air inlet section 40 and the air outlet section 42 are disposed on the insulated cover plate 43. The outdoor humidity measuring point 68 and the outdoor temperature measuring point 70 are provided in a spatial proximity to the air inlet section 40, so that a humidity and a temperature of the air flowing in via the air inlet section 40 can be detected.

FIG. 4 shows a top view of the mobile transport device without the cover plate of the housing 2. The receiving section 4 divides the interior 24 into an exhaust section 28 and a ventilation section 26. The direction of the arrow indicates that the recirculation fan 8 ventilates the air from the exhaust section 28 into the ventilation section 26, where it can be heated by the heating device 50. A CO2 measuring point 60 and a further outdoor temperature measuring point are also disposed in the surroundings, so that a CO2 concentration can be recorded in addition to a humidity and temperature of the incoming air.

FIG. 5 shows curves of heat generation in the different compartments 22.1-22.4 and thus of the insect larvae cohorts recorded therein at different times t1, t2, t3, t4, 15, 16 and t7, which are plotted on the abscissa axis. The points in time represent day 1, day 2, day 3, day 4, day 5, day 6 and day 7 of a joint transport of these compartments 22.1-22.4 with the mobile transport device 1. The insect larvae cohorts are of different ages, so that the individual heat generation curves in compartments 22.1-22.4 are shifted.

On the ordinate axis, the heat generation is plotted in watts in a range from 0 to 350 W.

The heat generation within the first compartment 22.1 and thus the first insect larvae cohort accommodated therein is approximately 25 watts at time t1, i.e. on the first day of transport, and remains almost constant until time t3. Heat generation increases from time t3 and reaches a maximum of approx. 120 watts shortly before time t6. The heat generation then drops again to approx. 20 watts by time t7. The heat generation process shows that the insect larvae picked up in the first compartment 22.2 are comparatively young insect larvae at the start of transport.

The heat generation within the second compartment 22.2 and thus the second cohort of insect larvae housed therein is approximately 10 watts at time t1, rising to approximately 45 watts by time t3 and then to approximately 120 watts between times t4 and t5. The heat generation then drops to approx. 10 watts by time t6. The heat generation curve shows that the insect larvae picked up in the second compartment 22.2 are comparatively older than the insect larvae picked up in the first compartment 22.1 at the start of transport.

The heat generation within the third compartment 22.3 and thus the third insect larvae cohort accommodated therein is approx. 20 watts at time t1, by time t2 the heat generation already increases to approx. 50 watts and then reaches a maximum of approx. 120 watts between times t3 and t4. The heat generation then drops to approx. 10 watts up to time t5 and remains constant up to time t7. The heat generation curve shows that the insect larvae collected in the third compartment 22.3 are comparatively older than the insect larvae collected in the first compartment 22.1 and the insect larvae collected in the second compartment 22.2 at the start of transport.

Within the fourth compartment 22.4, the heat generation is already approx. 45 watts at time t1. Between the times t2 and t3, heat generation already reaches a maximum of approx. 120 watts. The heat generation then drops to approx. 10 watts up to time t4 and remains constant at approx. 10 watts up to time t7. It can be seen from the progression that the insect larvae recorded in the fourth compartment 22.4 are the comparatively oldest insect larvae at the beginning of the transport.

Essentially, the curves of the individual compartments therefore show a phase shift of one day.

The heat generated by the insect larvae also produces energy that can be used to heat the circulating air. This can significantly reduce the energy consumption of the energy storage unit 74.

FIG. 5 also shows the course of an average heat generation with recirculation 96, which is achieved via the recirculation fan 8. At time t1, the average heat generation 96 with recirculation is approx. 20 watts; at time t2, it is already 50 watts. Between times t3 and t4, the average heat generation 96 reaches a maximum of approx. 75 watts and then remains almost constant at approx. 75 watts until time t5. The curve of average heat generation with recirculation then flattens out and drops to approx. 10 watts by time t7.

FIG. 5 also shows the course of a sum of the heat generation of compartments 22.1-22.4 without recirculation. The total heat generation without recirculation 98 is just under 100 watts at time t1, 200 watts at time t2 and then a maximum of approx. 290 watts at time t3. The course of the total heat generation without recirculation 98 drops to approx. 260 watts up to time t5, and then to just over 50 watts up to time t7.

The comparison between the average heat generation with recirculation 96 and the total heat generation without recirculation 98 shows that recirculation by means of the recirculation fan 8 results in a lower heat generation in the mobile transport device 1.

FIG. 6 shows curves of the ventilation demand of compartments 22.1-22.4, the average ventilation demand with recirculation 100 and the sum of the ventilation demand of compartments 22.1-22.4 without recirculation 102. On the abscissa axis are the times t1, t2, t3, t4, 5, t6 and t7, where, as in FIG. 5, the times represent day 1, day 2, day 3, day 4, day 5, day 6 and day 7 of the joint transport of these compartments 22.1-22.4 with the mobile transport device 1. On the ordinate axis, the ventilation requirement is listed in m³/h in a range from 0 m³/h to 20 m³/h. The calculated ventilation requirement according to FIG. 6 and the calculated heat generation according to FIG. 5 must be considered together.

The ventilation requirement of the first compartment 22.1 and thus of the insect larvae contained therein is slightly above 1 m³/h at time t1 and remains almost constant at 1 m³/h until time t3. The ventilation requirement of the first compartment 22.1 initially increases to 2 m³/h up to time t4 and then to 7 m³/h between times t5 and t6. The ventilation requirement then drops again to approx. 1 m³/h. The ventilation requirement of the first compartment 22.1 is determined by the heat generation of the first compartment 22.1 as shown in FIG. 6.

The ventilation requirement of the second compartment 22.2 is approx. 0.5 m³/h at time t1 and increases to 2 m³/h by time t3. Between the times t4 and t5, the ventilation requirement of the second compartment 22.2 and thus of the insect larvae housed in it reaches a maximum of 7 m³/h. By time t6, the ventilation requirement drops again to approx. 0.5 m³/h and remains constant until time t7. The ventilation requirement of the second compartment 22.2 is determined by the heat generation of the second compartment 22.2 as shown in FIG. 6.

The ventilation requirement of the third compartment 22.3 is just over 1 m³/h at time t1 and increases to 2 m³/h by time t2. A maximum ventilation requirement of 7 m³/h is required between the times t3 and t5. Up to time t7, the ventilation requirement of the third compartment 22.3 and thus of the insect larvae contained therein falls to approx. 0.5 m³/h. The ventilation requirement of the third compartment 22.3 is determined by the heat generation of the third compartment 22.3 as shown in FIG. 6.

The fourth compartment 22.4 already requires a ventilation requirement of over 2 m³/h at time t1. The ventilation requirement already reaches a maximum of 7 m³/h between the times t2 and t3. The ventilation requirement then drops to approx. 0.5 m³/h up to time t4 and remains constant up to time t7. The ventilation requirement of the fourth compartment 22.4 is determined by the heat generation of the fourth compartment 22.4 as shown in FIG. 6.

Here too, the curves of the individual compartments essentially show a phase shift of one day.

The average ventilation requirement of compartments 22.1-22.4 with recirculation 100 is just over 1 m³/h at time t1. Between times t3 and t4, the average ventilation requirement 100 reaches a maximum of just over 4 m³/h and then remains almost constant at around 4 m³/h until time t5. The curve of the average ventilation demand with recirculation 100 then flattens out and drops to approx. 0.5 m³/h by time t7.

The sum of the ventilation requirements of compartments 22.1-22.4 without recirculation 102 is approximately 6 m³/h at time t1, approximately 10 m³/h at time t2 and then a maximum of approximately 17 m³/h at time t3. Up to time t5, the total ventilation requirement without recirculation 102 initially falls to 16 m³/h, then to approx. 3 m³/h up to time t7.

The comparison between the average ventilation requirement with recirculation 100 and the sum of the ventilation requirement without recirculation 102 shows that recirculation by means of the recirculation fan 8 results in a lower ventilation requirement in the mobile transport device 1.

A mobile transport device 1 according to the second embodiment example of the invention is shown in FIG. 7. The second embodiment example of the mobile transport device 1 differs from the first embodiment example of the mobile transport device 1 (see FIG. 1) in that a cooling unit 51 is accommodated in the storage container 30. The other features of the second embodiment example of the mobile transport device 1 correspond to the features of the first embodiment example of the mobile transport device 1; identical and similar elements are therefore provided with the same reference signs. In this respect, full reference is made to the description above.

The cooling unit 51 is and/or comprises a cooling body which is an ice (water), a liquid nitrogen (nitrogen ice), a solid CO2 (a dry ice), a cooling compress such as, for example, a cool pack, a cooling pad, a Peltier element, a metallic and/or ceramic and/or mineral material or another cooling element and is configured to cool the insect larvae held in the insect fattening containers 6.1-6.4. The insect larvae can be cooled down so much that they are no longer active, i.e. they no longer move. As long as the insect larvae are to be kept in the cooled-down state, the heating device 50 is preferably switched off. By means of the heating device 50, however, it is possible to heat the insect larvae at any time and consequently return them to an active state. The cooling unit can also be or include a cooling unit for active cooling. The cooling unit for active cooling preferably comprises a fan, a pump and/or a compressor. The cooling unit for active cooling preferably comprises a coolant supply line for conducting coolant and a coolant discharge line for conducting coolant. Preferably, the coolant supply line and the coolant discharge line are connected at least via the fan, the pump or the compressor, with the coolant supply line preferably supplying coolant to the fan, the pump or the compressor and the coolant discharge line preferably discharging coolant from the fan, the pump or the compressor. Preferably, a coolant flows through the cooling unit for active cooling.

At the first insect fattening container temperature measurement point 56, an insect fattening container temperature measurement value can be detected. It can thus be checked whether the temperature in the insect fattening containers 6.1-6.4 is within a range that keeps the insect larvae in the cooled state.

FIG. 8 shows a schematic flow chart for a first preferred embodiment example of the method for transporting insect larvae with the mobile transport device 1, comprising filling the first insect fattening container 6.1 with insect larvae with the addition of fattening substrate (step S1), inserting the filled first insect fattening container 6.1 into the receiving section 4 of the mobile transport device 1 (step S2), transporting the insect larvae with the mobile transport device 1 from a first location to a second location (step S3) and removing the first insect fattening container 6.1 from the receiving section 4 at a second location (step S4).

FIG. 9 shows a schematic flow diagram for a second preferred embodiment example of the method for transporting insect larvae with the mobile transport device 1, which is a possible refinement of the first embodiment example of the method for transporting insect larvae (FIG. 8). During transport (step S3), it comprises providing signals from the activity sensor device 54 to the electronic control unit 10 (step S3.1.1), determining insect larval activity (step S3.1.2) and outputting control signals from the electronic control unit 10 to the recirculation fan 8 (step S3.1.3) based on the determination in step S3.1.2.

FIG. 10 shows a schematic flow diagram for a third preferred embodiment example of the method for transporting insect larvae with the mobile transport device 1, which is a possible refinement of the second embodiment example of the method for transporting insect larvae (FIG. 9). In addition to the actuation in step S3.1.3, it comprises a further outputting of control signals to the ventilation control unit (step 3.1.4) based on the determination in step S3.1.2.

FIG. 11 shows a schematic flow diagram for a fourth preferred embodiment example of the method for transporting insect larvae with the mobile transport device 1, which is a possible refinement of the first, second or third embodiment example (FIGS. 8, 9, 10) of the method for transporting insect larvae. During transport in step S3, it comprises providing signals from the air sensor device 60 to the electronic control unit 10 (step S3.2.1), determining an air state of air circulating in the housing 2 (step S3.2.2) and outputting control signals from the electronic control unit 10 to the storage container control unit 36 (step S3.2.3). Steps S3.1.1, S3.1.2, S3.1.3 and S3.1.4, which are also shown in FIG. 10, are optional.

FIG. 12 shows a schematic flow diagram for a fifth preferred embodiment example of the method for transporting insect larvae with the mobile transport device 1, which is a possible refinement of the first, second, third or fourth embodiment example (FIGS. 8, 9, 10, 11) of the method for transporting insect larvae. During transport in step S3, it comprises providing signals from the air sensor device 60 to the electronic control unit 10 (step S3.3.1), determining an air state of an ambient air (step S3.3.2) and outputting control signals from the electronic control unit 10 to the heating device 50 (step S3.3.3). Steps S3.1.1, S3.1.2, S3.1.3, S3.1.4, S3.2.1, S3.2.2 and S3.2.3, which are also illustrated in FIG. 10, are optional.

FIG. 13 shows a schematic flow diagram for a sixth preferred embodiment example of the method for transporting insect larvae with the mobile transport device 1, which is a possible refinement of the first, second, third, fourth or fifth embodiment example (FIGS. 8, 9, 10, 11, 12) of the method for transporting insect larvae. During the transport in step S3, it comprises providing signals from the air sensor device 60 to the electronic control unit 10 (step S3.4.1), determining a CO2 concentration measurement value overshoot (step S3.4.2) and a control signal output from the electronic control unit 10 to the fresh air fan 46 (step S3.4.3) and a control signal output from the electronic control unit 10 to the exhaust air fan 48 (step S3.4.4) in the event that a CO2 concentration measurement value overshoot has been determined. Steps S3.1.1, S3.1.2, S3.1.3, S3.1.4, S3.2.1, S3.2.2, S3.2.3, S3.3.1, S3.3.2 and S3.3.3, which are also illustrated in FIG. 10, are optional.

FIGS. 14A-14B show the activity of insect larvae, especially insect larvae of the black soldier fly, as the insect larvae develop, as known from repeated observation of the insect larvae. At the beginning of a fattening phase, the insect larvae are evenly distributed in the first insect fattening container 6.1 (see FIG. 14A). The insect fattening container is completely filled with fattening substrate (see FIG. 14B).

As development progresses and activity increases, the insect larvae group together in a central section 82 of the insect fattening container 6.1 (see FIG. 14C). The fattening substrate increasingly dries out and at this point essentially only covers the bottom of the insect fattening container 6.1 (see FIG. 14D).

FIGS. 14E and 14F show the distribution of insect larvae at a later stage of development. The insect larvae are now also grouped in the corners of the cuboid insect fattening container 6.1 (see FIG. 14E). The side view as shown in FIG. 14F shows that the insect larvae are not grouped flat on the floor, but are essentially clustered over the entire height of the insect fattening container 6.1.

FIG. 15 shows a stationary insect larvae rearing device 78. A first insect fattening container 6.1, a second insect fattening container 6.2 and further insect fattening containers are disposed in the rearing device. The insect fattening containers are stacked vertically in three rows as shown in FIG. 15.

An activity sensor device 54 is provided in each of the insect fattening containers 6.1, 6.2 for detecting the activity of the insect larvae held in the respective insect fattening container 6.1, 6.2. The measured values recorded by the activity sensor device 54 are provided to the electronic control unit 10 and thus to a processing unit 80 integrated in the electronic control unit 10. Furthermore, measurement data of an air sensor device 60 is provided at the electronic control unit 10, wherein the air sensor device 60 can detect an air condition both inside and outside the insect larvae rearing device 78. The electronic control unit 10 is also connected to a computer 108 so that the measured values processed by the processing unit 80 can be displayed for a user.

In addition, a recirculation fan 8, a heating device 50 and a humidifier 76, which can be controlled by the electronic control unit 10, are disposed within the insect larvae rearing device 78.

FIG. 16 shows the arrangement of the activity sensor device 54 within the first insect fattening container 6.1. A first insect fattening container temperature measuring point 56 and a first insect fattening container humidity measuring point 58 are disposed in a central section 82 of the first insect fattening container 6.1. A second insect fattening container temperature measuring point 88 and a second insect fattening container humidity measuring point 84 are disposed in close proximity to one another on a side wall of the first insect fattening container 6.1. A third insect fattening container temperature measuring value 90 and a third insect fattening container humidity measuring value 86 are disposed at a corner of the insect fattening container 6.1. According to FIG. 16, the second and third insect fattening container humidity measurement values 84, 86 also extend over the height of the insect fattening container 6.1.

Based on the observed activity according to FIGS. 14A-14F, it can be assumed that the insect larvae cluster during their development first in the central section 82, i.e. at the first insect fattening container temperature measuring point 56 and at the first insect fattening container humidity measuring point 58, and then additionally at the third insect fattening container temperature measuring point 90 and at the third insect fattening container humidity measuring point 86. However, at the second insect fattening container temperature measuring point 88 and the second insect fattening container humidity measuring point, the insect larvae according to FIGS. 14A-14F will not group together.

FIG. 17 also shows an arrangement of the activity sensor device 54 within the first insect fattening container 6.1, but now having sensors instead of measuring points. A first humidity sensor 92.1 is disposed at the first insect fattening container humidity measuring point 58 (cf. FIG. 16), a second humidity sensor 92.2 is disposed at the second insect fattening container humidity measuring point 84 (cf. FIG. 16) and a third humidity sensor 92.3 is disposed at the third insect fattening container humidity measuring point 86 (cf. FIG. 16).

Furthermore, a first temperature sensor 94.1 is disposed at the first insect fattening container temperature measuring point 56 (cf. FIG. 16), a second temperature sensor 94.2 is disposed at the second insect fattening container temperature measuring point 88 (cf. FIG. 16) and a third temperature sensor 94.3 is disposed at the third insect fattening container temperature measuring point 90 (cf. FIG. 16).

The sensors 92.1-92.4, 94.1-94.4 provide signals representing the detected measured values 56, 58, 84, 86, 88, 90 to the electronic control unit 10 and consequently to the processing unit 80.

FIG. 18 shows curves of the measured values recorded by the humidity and temperature sensors as well as the fattening substrate humidity 104 at the times t0, t1, t2, t3, t4, 15, t6, t7 and t8. The points in time are plotted on the abscissa axis. The left-hand ordinate shows the humidity in percent in a range from 0% to 120%. The temperature in ° C. in a range from 20° C. to 40° C. is plotted on the right-hand ordinate.

The fattening substrate humidity 104, which can be regarded as a reference value for the measured humidity values recorded by the humidity sensors, is 80% at time t1, approximately 70% at time t4 and 40% at time t7. Accordingly, the fattening substrate humidity decreases by 40% between the times t1 and t7.

The first humidity sensor 92.1 disposed in the central section 82 (cf. FIG. 17) detects a humidity substantially corresponding to the fattening substrate humidity 104 up to the time t4. From time t4, the humidity detected by the first humidity sensor 92.1 begins to deviate from the fattening substrate humidity 104 and rises to 100% humidity by time t7. It can therefore be assumed that the insect larvae cluster at the first humidity sensor 92.1 from time t4, which means that the measured values recorded represent not only the fattening substrate humidity, but also the additional humidity of the insect larvae.

The second humidity sensor 92.2 with an arrangement according to FIG. 17 detects a humidity that substantially corresponds to the fattening substrate humidity 104. It can therefore be assumed that the insect larvae do not cluster at the second humidity sensor 92.2.

The third humidity sensor 92.3 with an arrangement according to FIG. 17 detects a humidity that essentially corresponds to the fattening substrate humidity 104 up to the time t5. Up to time t6, the humidity initially rises to approx. 65% and then to 90% up to time t7. It can therefore be assumed that the insect larvae cluster at the third humidity sensor 92.3 from time t5, which means that the measured values recorded represent not only the fattening substrate humidity, but also the additional humidity of the insect larvae.

The second temperature sensor 94.2 essentially records a constant temperature of 28° C. over the times t1-t7. Only at time t4 does the second temperature sensor 94.2 detect a temperature of 30° C.

The first temperature sensor 94.1 also detects an essentially constant temperature of approx. 28° C. up to time t3. The recorded temperature then rises to around 33° C. by time t4 and finally to 38° C. by time t5. This rise in temperature is due to an increase in activity and an associated increase in heat emission from the insect larvae, which cluster at the first temperature sensor 94.1. The recorded temperature then drops to approx. 32° C. at times t6 and t7.

The third temperature sensor 94.3 also detects an essentially constant temperature of approx. 28° C. up to time t3. The recorded temperature then rises to around 33° C. by time t4 and finally to 38° C. by time t5. This rise in temperature is due to an increase in activity and an associated increase in heat emission from the insect larvae, which cluster at the third temperature sensor 94.3. The recorded temperature then initially drops to approx. 34° C. at time t6 and then rises slightly to 35° C. by time t7.

FIG. 19 shows a schematic flow chart for a first preferred embodiment example of the method for determining an activity of insect larvae with the insect larvae rearing device 78, comprising filling the first insect fattening container 6.1 with insect larvae with the addition of fattening substrate at the beginning of a fattening phase (step SI) and processing the measured values recorded by means of the activity sensor device 54 with the processing unit 80 at a first time t1 (step SII.1). Processing at time t1 in step SII. 1 preferably comprises the following steps: Comparison of the recorded measured values with reference values at time t1 (step A1), determination of a reference value shortfall at time t1 (step B1), determination of a reference value overshoot at time t1 (step C1), determination of a cluster formation at time t1 (step D1), Comparing the cluster formation determined in step D1 with a reference cluster formation at the time t1 (step E1), determining a regular activity of the insect larvae at the time t1 (step F1), determining an irregular activity of the insect larvae at the time t1 (step G1) and outputting a developmental state signal at the time t1 (step H1).

FIG. 20 shows a schematic flow chart for a second preferred embodiment example of the method for determining an activity of insect larvae, which is a possible refinement of the first embodiment example of the method for determining an activity of insect larvae (FIG. 19).

In this second preferred embodiment method, the processing at time t1 in step SII.1 is followed by processing of the measured values recorded by the activity sensor device 54 with the processing unit 80 at the second time t2 in step SII.2. Processing at time t2 (step SII.2) comprises steps A2-H2, which correspond to steps A1-H1, but are carried out for time t2.

The processing at time t2 in step SII.2 is followed by processing of the measured values recorded by the activity sensor device 54 with the processing unit 80 at the third time t3 in step SII.2. Processing at time t3 (step SII.3) comprises steps A3-H3, which correspond to steps A1-H1 and A2-H2, but are carried out at time t3.

The processing at time t2 in step SII.2 is followed by processing of the measured values recorded by the activity sensor device 54 with the processing unit 80 at a third time t3 in step SII.3. Processing at time t3 (step SII.3) comprises steps A3-H3, which correspond to steps A1-H1 and A2-H2, but are carried out at time t3.

The processing at time t3 in step SII.3 is followed by processing of the measured values recorded by the activity sensor device 54 with the processing unit 80 at a third time t4 in step SII.4. Processing at time t4 (step SII.4) comprises steps A4-H4, which correspond to steps A1-H1, A2-H2 and A3-H3, but are carried out at time t4.

The processing at time t4 in step SII.4 is followed by processing of the measured values recorded by the activity sensor device 54 with the processing unit 80 at a third time t5 in step SII.5. Processing at time t5 (step SII.5) comprises steps A5-H5, which correspond to steps A1-H1, A2-H2, A3-H3 and A4-H4, but are carried out at time t5.

The processing at time t5 in step SII.5 is followed by processing of the measured values recorded by the activity sensor device 54 with the processing unit 80 at a third time t6 in step SII.6. Processing at time t6 (step SII.6) comprises steps A6-H6, which correspond to steps A1-H1, A2-H2, A3-H3. A4-H4 and A5-H5, but are carried out at time t6.

The processing at time t6 in step SII.6 is followed by processing of the measured values recorded by the activity sensor device 54 with the processing unit 80 at a third time t7 in step SII.7. Processing at time t7 (step SII.7) comprises steps A7-H7, which correspond to steps A1-H1, A2-H2, A3-H3, A4-H4, A5-H5, A6-H6, but are carried out at time t7.

FIG. 21 shows an embodiment example of a mobile insect larvae rearing device 110, which can also be used as a mobile transport device 1. The embodiment example is based on the embodiment example of the mobile insect transport device 1 and the same and similar elements are provided with the same reference signs as in the first embodiment example. In this respect, full reference is made to the description above. A first insect fattening container 6.1, a second insect fattening container 6.2, and further insect fattening containers are accommodated in the mobile insect larvae rearing device 110. The insect fattening containers are stacked in two rows as shown in FIG. 21. An activity sensor device 54 is provided in each of the insect fattening containers 6.1, 6.2. The measured values recorded by the activity sensor device 54 may be provided to the electronic control unit 10 and to the processing unit 10 integrated therein.

The energy storage unit 74 is connected to the electronic control unit 10 in order to supply it with electrical energy. In the embodiment example according to FIG. 21, a heating device 50 is disposed in a lower section of the mobile insect larvae rearing device 110 in such a way that the insect fattening containers 6.1, 6.2 can be positioned above the heating device 50. In the mobile insect larvae rearing device 110, two recirculation fans 8 are also provided for recirculating air within the mobile insect larvae rearing device 110.

FIG. 22 shows a further embodiment example of the mobile insect larvae rearing device 110. In contrast to the embodiment example shown in FIG. 20, four recirculation fans 8 are provided for recirculating air within the mobile insect larvae rearing device 110. In addition, the measured values recorded by the activity sensor device 54 can be provided wirelessly to the electronic control unit 10 and thus wirelessly to the processing unit 80.

1. A black soldier fly larvae rearing device, comprising:
a first insect fattening container configured to accommodate a first cohort of insect larvae for fattening;
an activity sensor device; and
a processing unit,
wherein the activity sensor device is configured to detect at least one first humidity measurement value at a first insect fattening container humidity measurement point and to provide the at least one first humidity measurement value to the processing unit,
wherein the at least one first humidity measurement point is disposed in a central portion of the first insect fattening container,
wherein the activity sensor device is configured to detect at least one first temperature measurement value at a first insect fattening container temperature measurement point and to provide the at least one first temperature measurement value to the processing unit,
wherein the at least one first temperature measurement point is disposed in the central portion of the first insect fattening container, and
wherein the processing unit is configured to process the detected first humidity measurement value and the detected first temperature measurement value and to determine an activity of the insect larvae based on the processing.

2. The rearing device of claim 1, wherein:
the activity sensor device is configured to detect at least one second humidity measurement value at a second insect fattening container humidity measurement point and to provide the at least one second humidity measurement value to the processing unit,
the second insect fattening container humidity measurement point is disposed laterally spaced from the first insect fattening container humidity measurement point,
the activity sensor device is configured to detect at least one second temperature measurement value at a second insect fattening container temperature measurement point and to provide the at least one second temperature measurement value to the processing unit, and
the second insect fattening container temperature measurement point is disposed laterally from the first insect fattening container temperature measurement point.

3. The rearing device of claim 2, wherein:
the activity sensor device is configured to detect at least one third humidity measurement value at a third insect fattening container humidity measurement point and to provide the at least one third humidity measurement value to the processing unit,
the third insect fattening container humidity measurement point is disposed laterally from both the first insect fattening container humidity measurement point and the second insect fattening container humidity measurement point,
the activity sensor device is configured to detect at least one third temperature measurement value at a third insect fattening container temperature measurement point and to provide the at least one third temperature measurement value to the processing unit, and
the third insect fattening container temperature measurement point is disposed laterally spaced from both the first insect fattening container temperature measurement point and the second insect fattening container temperature measurement point.

4. The rearing device of claim 3, wherein;
the first insect fattening container humidity measuring point is disposed adjacent to the first insect fattening container temperature measuring point,
the second insect fattening container humidity measuring point is disposed adjacent to the second insect fattening container temperature measuring point, and/or
the third insect fattening container humidity measuring point is disposed adjacent to the third insect fattening container temperature measuring point.

5. The rearing device of claim 3, wherein;
a first humidity sensor is disposed at the first insect fattening container humidity measuring point,
a second humidity sensor is disposed at the second insect fattening container humidity measuring point, and/or
a third humidity sensor is disposed at the third insect fattening container humidity measuring point.

6. The rearing device of claim 3, wherein;
a first temperature sensor is disposed at the first insect fattening container temperature measuring point,
a second temperature sensor is disposed at the second insect fattening container temperature measuring point, and/or
a third temperature sensor is disposed at the third insect fattening container temperature measuring point.

7. The rearing device of claim 1, further comprising a second insect fattening container configured to accommodate a second insect larvae cohort for fattening.

8. The rearing device of claim 7, wherein:
the activity sensor device is configured to detect at least one first humidity measurement value at a first insect fattening container humidity measurement point of the second insect fattening container and to provide the at least one first humidity measurement value to the processing unit,
the first insect fattening container humidity measuring point of the second insect fattening container is disposed in a central portion of the second insect fattening container,
the activity sensor device is configured to detect at least one first temperature measurement value at a first insect fattening container temperature measuring point of the second insect fattening container and to provide the at least one first temperature measurement value to the processing unit,
the first insect fattening container temperature measuring point is disposed in the central portion of the second insect fattening container,
the processing unit is configured to process the detected first humidity measurement value of the second insect fattening container and the detected first temperature measurement value of the second insect fattening container and to determine an activity of the insect larvae in the second insect fattening container based on the processing.

9. The rearing device of claim 8, wherein:
the activity sensor device is configured to detect at least one second humidity measurement value at a second insect fattening container humidity measuring point of the second insect fattening container and to provide the at least one second humidity measurement value to the processing unit,
the second insect fattening container humidity measuring point of the second insect fattening container is disposed laterally from the first insect fattening container humidity measuring point of the second insect fattening container,
the activity sensor device is configured to detect at least one second temperature measurement value at a second insect fattening container temperature measuring point of the second insect fattening container and to provide the at least one second temperature measurement value to the processing unit, and
the second insect fattening container temperature measuring point of the second insect fattening container is disposed laterally from the first insect fattening container temperature measuring point of the second insect fattening container.

10. The rearing device of claim 9, wherein:
the activity sensor device is configured to detect at least one third humidity measurement value at a third insect fattening container humidity measuring point of the second insect fattening container and to provide the at least one third humidity measurement value to the processing unit,
the third insect fattening container humidity measuring point of the second insect fattening container is disposed laterally from both the first insect fattening container humidity measuring point of the second insect fattening container and the second insect fattening container humidity measuring point of the second insect fattening container,
the activity sensor device is configured to detect at least one third temperature measurement value at a third insect fattening container temperature measuring point of the second insect fattening container and to provide the at least one third temperature measurement value to the processing unit, and
the third insect fattening container temperature measuring point of the second insect fattening container is disposed laterally from both the first insect fattening container temperature measuring point of the second insect fattening container and the second insect fattening container temperature measuring point of the second insect fattening container.

11. A method for determining activity of black soldier fly larvae, the method comprising:
filling a first insect fattening container with insect larvae with and fattening substrate at the start of a fattening phase; and
processing a detected first humidity measurement value and a detected first temperature measurement value at a first time t1 by:
A1) Comparing the detected first humidity measurement value at time t1 with a first humidity reference value at time t1, comparing the detected first temperature measurement value at time t1 with a first temperature reference value at time t1;
B1) Determining a reference value undershoot at time t1 in the event that one or more of the detected measurement values falls below the respective reference value;
C1) Determining a reference value overshoot at time t1 in the event that one or more of the detected measurement values exceed the respective reference value;
D1) Determining a cluster formation of the insect larvae at time t1 at one or more of the humidity and/or temperature measuring points in the event that a reference value overshoot was detected at said one or more humidity and/or temperature measuring points at time t1;
E1) Comparing the determined cluster formation of the insect larvae at time t1 with a reference cluster formation of the insect larvae at time t1;
F1) Determining a regular activity of the insect larvae at time t1 in the event that the determined cluster formation of the insect larvae at time t1 corresponds to the reference cluster formation of the insect larvae at time t1;
G1) Determining an irregular activity of the insect larvae at time t1 in the event that the determined cluster formation of the insect larvae at time t1 deviates from the reference cluster formation of the insect larvae at time t1; and
H1) Outputting a development status signal at time t1 depending on the determined activity of the insect larvae at time t1.

12. The method of claim 11, further comprising:
processing a detected second humidity measurement value and a detected second temperature measurement value at the first time t1, wherein the step A1) further comprises:
comparing the detected second humidity measurement value at time t1 with a second humidity reference value at time t1, and
comparing the detected second temperature measurement value at time t1 with a second temperature reference value at time t1.

13. The method of claim 12, further comprising:
processing a detected third humidity measurement value and a detected third temperature measurement value at the first time t1, wherein the step A1) further comprises:
comparing the detected third humidity measurement value at time t1 with a third humidity reference value at time t1, and
comparing the detected third temperature measurement value at a time t1 with a third temperature reference value at time t1.

14. The method of claim 13, further comprising:
Processing of processing the detected first, second, and/or third humidity measurement values and of the detected first, second, and/or third temperature measurement values at a second time t2, at a third time t3, at a fourth time t4, at a fifth time t5, at a sixth time t6, and/or at a seventh time t7, wherein:
a time period of from one hour to 48 hours lies between time t1 and time t2,
a time period of from one hour to 48 hours lies between time t2 and time t3,
a time period of from one hour to 48 hours lies between time t3 and time t4,
a time period of from one hour to 48 hours lies between time t4 and time t5,
a time period of from one hour to 48 hours lies between time t5 and time t6, and/or
a time period of from one hour to 48 hours lies between time t6 and time t7,
the method further comprises: comprising the steps:
A2) comparing the detected first humidity measurement value at time t2 with a first humidity reference value at time t2, comparing the detected second humidity measurement value at time t2 with a second humidity reference value at time t2, comparing the detected third humidity measurement value at time t2 with a third humidity reference value at time t2, comparing the detected first temperature measurement value at time t2 with a first temperature reference value at time t2, comparing the detected second temperature measurement value at time t2 with a second temperature reference value at time t2 and comparing the detected third temperature measurement value at time t2 with a third temperature reference value at time t2;
B2) determining a reference value undershoot at time t2 in the if one or more of the detected measurement values falls below the respective reference value;
C2) determining a reference value overshoot at time t2 in the event that if one or more of the detected measurement values exceed the respective reference value;
D2) determining a cluster formation of the insect larvae at time t2 at one or more of the humidity and/or temperature measuring points in the event that if a reference value overshoot was identified at said one or more humidity and/or temperature measuring points at time t2;
E2) comparing the determined cluster formation of the insect larvae at time t2 with a reference cluster formation of the insect larvae at time t2;
F2) determining a regular activity of the insect larvae at time t2 in the event that if the determined cluster formation of the insect larvae at time t2 corresponds to the reference cluster formation of the insect larvae at time t2;
G2) determining an irregular activity of the insect larvae at time t2 in the event that if the determined cluster formation of the insect larvae at time t2 deviates from the reference cluster formation of the insect larvae at time t2; and
H2) outputting a development status signal at time t2 depending on the determined activity of the insect larvae at time t2;
A3) comparing the detected first humidity measurement value at time t3 with a first humidity reference value at time t3, comparing the detected second humidity measurement value at time t3 with a second humidity reference value at time t3, comparing the detected third humidity measurement value at time t3 with a third humidity reference value at time t3, comparing the detected first temperature measurement value at time t3 with a first temperature reference value at time t3, comparing the detected second temperature measurement value at time t3 with a second temperature reference value at time t3, and comparing the detected third temperature measurement value at time t3 with a third temperature reference value at time t3;
B3) determining a reference value undershoot at time t3 if one or more of the detected measurement values falls below the respective reference value;
C3) determining a reference value overshoot at time t3 if one or more of the detected measurement values exceed the respective reference value;
D3) determining a cluster formation of the insect larvae at time t3 at one or more of the humidity and/or temperature measuring points if a reference value overshoot was detected at said one or more humidity and/or temperature measuring points at time t3;
E3) comparing the determined cluster formation of the insect larvae at time t3 with a reference cluster formation of the insect larvae at time t3;
F3) determining a regular activity of the insect larvae at time t3 if the determined cluster formation of the insect larvae at time t3 corresponds to the reference cluster formation of the insect larvae at time t3;
G3) determining an irregular activity of the insect larvae at time t3 if the determined cluster formation of the insect larvae at time t3 deviates from the reference cluster formation of the insect larvae at time t3; and
H3) outputting a development status signal at time t3 depending on the determined activity of the insect larvae at time t3;
A4) comparing the detected first humidity measurement value at time t4 with a first humidity reference value at time t4, comparing the detected second humidity measurement value at time t4 with a second humidity reference value at time t4, comparing the detected third humidity measurement value at time t4 with a third humidity reference value at time t4, comparing the detected first temperature measurement value at time t4 with a first temperature reference value at time t4, comparing the detected second temperature measurement value at time t4 with a second temperature reference value at time t4 and comparing the detected third temperature measurement value at time t4 with a third temperature reference value at time t4;
B4) determining a reference value undershoot at time t4 if one or more of the detected measurement values falls below the respective reference value;
C4) determining a reference value overshoot at time t4 if one or more of the detected measurement values exceed the respective reference value;
D4) determining a cluster formation of the insect larvae at time t4 at one or more of the humidity and/or temperature measuring points if a reference value overshoot was detected at said one or more humidity and/or temperature measuring points at time t4;
E4) comparing the determined cluster formation of the insect larvae at time t4 with a reference cluster formation of the insect larvae at time t4;
F4) determining a regular activity of the insect larvae at time t4 if the determined cluster formation of the insect larvae at time t4 corresponds to the reference cluster formation of the insect larvae at time t4;
G4) determining an irregular activity of the insect larvae at time t4 if the determined cluster formation of the insect larvae at time t4 deviates from the reference cluster formation of the insect larvae at time t4; and
H4) outputting a development status signal at time t4 depending on the determined activity of the insect larvae at time t4;
A5) comparing the detected first humidity measurement value at time t5 with a first humidity reference value at time t5, comparing the detected second humidity measurement value at time t5 with a second humidity reference value at time t5, comparing the detected third humidity measurement value at time t5 with a third humidity reference value at time t5, comparing the detected first temperature measurement value at time t5 with a first temperature reference value at time t5, comparing the detected second temperature measurement value at time t5 with a second temperature reference value at time t5, and comparing the detected third temperature measurement value at time t5 with a third temperature reference value at time t5;
B5) determining a reference value undershoot at time t5 if one or more of the detected measurement values falls below the respective reference value;
C5) determining a reference value overshoot at time t5 if one or more of the detected measurement values exceed the respective reference value;
D5) determining a cluster formation of the insect larvae at time t5 at one or more of the humidity and/or temperature measuring points if a reference value overshoot was detected at said one or more humidity and/or temperature measuring points at time t5;
E5) comparing the determined cluster formation of the insect larvae at time t5 with a reference cluster formation of the insect larvae at time t5;
F5) determining a regular activity of the insect larvae at time t5 if the determined cluster formation of the insect larvae at time t5 corresponds to the reference cluster formation of the insect larvae at time t5;
G5) determining an irregular activity of the insect larvae at time t5 in the event that if the determined cluster formation of the insect larvae at time t5 deviates from the reference cluster formation of the insect larvae at time t5; and
H5) outputting a development status signal at time t5 depending on the determined activity of the insect larvae at time t5;
A6) comparing the detected first humidity measurement value at time t6 with a first humidity reference value at time t6, comparing the detected second humidity measurement value at time t6 with a second humidity reference value at time t6, comparing the detected third humidity measurement value at time t6 with a third humidity reference value at time t6, comparing the detected first temperature measurement value at time t6 with a first temperature reference value at time t6, comparing the detected second temperature measurement value at time t6 with a second temperature reference value at time t6, and comparing the detected third temperature measurement value at time t6 with a third temperature reference value at time t6;
B6) determining a reference value undershoot at time t6 if one or more of the detected measurement values falls below the respective reference value;
C6) determining a reference value overshoot at time t6 if one or more of the detected measurement values exceed the respective reference value;
D6) determining a cluster formation of the insect larvae at time t6 at one or more of the humidity and/or temperature measuring points if a reference value overshoot was detected at said one or more humidity and/or temperature measuring points at time t6;
E6) comparing the determined cluster formation of the insect larvae at time t6 with a reference cluster formation of the insect larvae at time t6;
F6) determining a regular activity of the insect larvae at time t6 if the determined cluster formation of the insect larvae at time t6 corresponds to the reference cluster formation of the insect larvae at time t6;
G6) determining an irregular activity of the insect larvae at time t6 if the determined cluster formation of the insect larvae at time t6 deviates from the reference cluster formation of the insect larvae at time t6; and
H6) outputting a development status signal at time t6 depending on the determined activity of the insect larvae at time t6; and/or
A7) comparing the detected first humidity measurement value at time t7 with a first humidity reference value at time t7, comparing the detected second humidity measurement value at time t7 with a second humidity reference value at time t7, comparing the detected third humidity measurement value at time t7 with a third humidity reference value at time t7, comparing the detected first temperature measurement value at time t7 with a first temperature reference value at time t7, comparing the detected second temperature measurement value at time t7 with a second temperature reference value at time t7, and comparing the detected third temperature measurement value at time t7 with a third temperature reference value at time t7;
B7) determining a reference value undershoot at time t7 if one or more of the detected measurement values falls below the respective reference value;
C7) determining a reference value overshoot at time t7 if one or more of the detected measurement values exceed the respective reference value;
D7) determining a cluster formation of the insect larvae at time t7 at one or more of the humidity and/or temperature measuring points if a reference value overshoot was detected at said one or more humidity and/or temperature measuring points at time t7;
E7) comparing the determined cluster formation of the insect larvae at time t7 with a reference cluster formation of the insect larvae at time t7;
F7) determining a regular activity of the insect larvae at time t7 if the determined cluster formation of the insect larvae at time t7 corresponds to the reference cluster formation of the insect larvae at time t7;
G7) determining an irregular activity of the insect larvae at time t7 if the determined cluster formation of the insect larvae at time t7 deviates from the reference cluster formation of the insect larvae at time t7; and
H7) outputting a development status signal at time t7 depending on the determined activity of the insect larvae at time t7.

15. The method of claim 11, further comprising-continuing the fattening phase if the developmental status signal indicates regular activity of the insect larvae.

16. The method of claim 11, further comprising-interrupting the fattening phase if the developmental status signal indicates irregular activity of the insect larvae.

17. The method of claim 14, further comprising
determining an average temperature based on the first, second, and/or third temperature measurement values at times t1, t2, t3, t4, t5, t6 and/or t7;
determining a fattening substrate evaporation rate based on the determined average temperatures and a predetermined humidity; and/or
determining a dry matter content of the fattening substrate based on the determined fattening substrate evaporation.

18. A computer program comprising program code which, when executed on a processing unit of a black soldier fly larvae rearing device causes the processing unit to execute the method of claim 11.
